(19) United States
(12) Patent Application Publication
(10) Pub. No.: US 2020/0141935 A1
(43) Pub. Date: May 7, 2020

(54) SYSTEM, METHOD, APPARATUS AND DIAGNOSTIC TEST FOR PREGNANCY

(71) Applicant: BARCELONA INSTITUTE FOR GLOBAL HEALTH, Barcelona (ES)

(72) Inventor: Alfredo MAYOR, Barcelona (ES)

(21) Appl. No.: 16/624,998

(22) PCT Filed: Jun. 25, 2018
(86) PCT No.: PCT/IB2018/054656

§ 371 Date: Dec. 20, 2019

Related U.S. Application Data
(60) Provisional application No. 62/523,828, filed on Jun. 23, 2017.

(51) Int. Cl. G01N 33/569; G01N 33/563; C07K 16/20

(52) U.S. Cl. CPC G01N 33/56905; G01N 2333/445; C07K 16/205; G01N 33/563

(57) ABSTRACT

The present invention, in at least some embodiments, is of a system, method, apparatus and diagnostic test for monitoring infections by Plasmodium falciparum that is specific for pregnant women. The monitoring is performed by examining samples from the pregnant women, typically blood samples, for the presence of antibodies to a known P. falciparum protein, VAR2CSA. Preferably, the antibodies bind specifically to p5 and/or p8.

### FIELD OF THE INVENTION

The present invention is of a system, method, apparatus and diagnostic test for pregnancy specific serologic monitoring of Plasmodium species, and in particular, to such a system, method, apparatus and diagnostic test for monitoring infections by Plasmodium falciparum that is specific for pregnant women.

### BACKGROUND OF THE INVENTION

RBC (red blood cells) infected by the late developmental stages of P. falciparum blood parasites are not found in the peripheral circulation, as they adhere to receptors on the endothelial lining. This adhesion, called sequestration, is mediated through parasite-encoded, clonally variant surface antigens (VSA) inserted into the membrane of the infected RBC (IRBC) and is thought to be an immune evasion strategy, possibly evolved to avoid splenic clearance.

The best-characterized VSA are encoded by the var genes. This gene family, encompassing about 60 members per genome, encodes the variant protein P. falciparum erythrocyte membrane protein 1 (PfEMP1), which is located on the surface of the P. falciparum-infected erythrocytes where it mediates adhesion.

A given parasite expresses only one PfEMP1 at a time, but in each generation a fraction of the daughter parasites may switch to expression of alternative PfEMP1 species through an unknown process. Different PfEMP1 molecules have different receptor specificities, and clonal switching between expression of the various var gene products in a mutually exclusive manner allows the parasite to modify its adhesion properties, which in turn determines in which tissue the parasite can sequester.

Plasmodium falciparum infection during pregnancy is associated with parasitized erythrocyte (PE) sequestration in the placenta, and contributes to low birthweight babies and neonatal mortality (Brabin B. J. et al. 2004 Placenta 25:359-378). Placental isolates are functionally distinct because they do not bind CD36, but instead bind chondroitin sulphate A (CSA) (Fried M. & Duffy P. E. 1996 Science 272:1502-1504). US20090130136 to Miller et al demonstrated that VAR2CSA does include CSA binding domains and so binds to CSA. CSA is abundant in the placenta but not in any other organ. P. falciparum parasites that infect pregnant women do so through the placenta and are therefore generally only able to effectively infect pregnant women.

Malaria infected pregnant women develop antibodies against P. falciparum erythrocyte membrane protein VAR2CSA (350 kDa) that binds to CSA in the syncytiotrophoblasts [9]. As an erythrocyte stage protein, VAR2CSA is less suitable as a target for vaccine production, as it cannot block new or further infections, as noted for example in EP2548572A2 to German Perez et al. Nonetheless, as VAR2CSA is specific to pregnant women, it has been considered for development of vaccines against P. falciparum that are directed toward pregnant women (see for example U.S. Pat. No. 9,540,425 to Ndam et al).

In recent years, there has been a decline in malaria transmission in many regions, leading to optimism that malaria elimination might be achieved in numerous countries (WHO 2016). As transmission declines, surveillance becomes increasingly important and metrics used to estimate malaria exposure in a community need to account for dynamic changes over space and time essential to guide strategic planning, implementation and evaluation of interventions.

Traditionally, surveillance has been typically reliant on case reporting by health services, entomological estimates and parasitemia point prevalence. However, these metrics are difficult to apply, costly and poorly informative as transmission declines towards elimination. In contrast, serology has recently become more attractive as an epidemiologic tool [1,2] and discovery of new antigenic targets is a research priority on the malaria elimination agenda (MalERA) enhanced by the improved technology for high-throughput screening [3,4]. The currently used sero-surveillance assays (community-based seroconversion rates) [5] are not designed to detect short-term or gradual changes in P. falciparum exposure at an individual level. This is mainly due to the quick acquisition of antibody responses and the long half-life reported to the readily available blood-stage antigens. This reinforces the idea that sero-surveillance tools can be improved by selecting new antigens less immunogenic and more short-lived [3,4,6]. Furthermore, cross-sectional household-based surveys are time-consuming, operationally demanding, and costly, and once transmission becomes low, the sample sizes required make them unfeasible for the purposes of routine surveillance [7]. To overcome this limitation, routine surveillance can approach easily accessible groups that are particularly sensitive to changes in transmission and representative of the malaria burden in the community (i.e school children or pregnant women attending antenatal services) [8].

Antibodies to VAR2CSA are developed in a parity dependent manner (i.e., increase with exposure during successive pregnancies) [10] and are affected by variables that influence the risk of exposure to P. falciparum such as season, proximity to the river [11], use of IPTp [12] or insecticide-treated nets [ITN] [13]. Relatively low serological diversity of VAR2CSA [14] and development of antibodies after single or very limited exposures to placental parasites [15] supports the suitability of this antigen for the serological estimation of transmission. Moreover, P. falciparum prevalence in pregnant women was shown to strongly correlate with prevalence of infection detected in children [8,16].

### BRIEF SUMMARY OF THE INVENTION

The present invention, in at least some embodiments, is of a system, method, apparatus and diagnostic test for monitoring infections by Plasmodium falciparum that is specific for pregnant women. The monitoring is performed by examining samples from the pregnant women, typically blood samples, for the presence of antibodies to a known P. falciparum protein, VAR2CSA. Preferably, the antibodies bind specifically to p5 and/or p8.

p5 (aa):
(SEQ ID NO: 1)
CRKCGHYEEKVPTKLDYVPQFLRWLTEWIEDLYREKQNLIDDMERHREECT
p8 (aa):
(SEQ ID NO: 2)
DEVCNCNESEISSVGQAQTSGPSSNKTCITHSSIKANKKKVCKDVKLG

Surprisingly, the present inventors have found that antibodies to VAR2CSA have widely varying half-lives. Certain antibodies have relatively long half-lives, meaning that the presence of such antibodies may in fact indicate an exposure during an earlier pregnancy, rather than during the current pregnancy. However there are clear benefits to determining whether an exposure occurred during a current pregnancy in women. Without wishing to be limited by a closed list, these benefits include being able to track exposure to malaria in an overall population, which may for example give guidance to control and eradication efforts, including estimating the level of malaria burden/transmission, as a proxy for parasite prevalence in the community and monitoring the absence of malaria transmission; tracking such exposure to pregnant women in particular, as despite of the increased risk to malaria, many antimalarial drugs are not recommended during early pregnancy due to safety concerns for the fetus; directing medical efforts toward assisting children born after exposure to a malarial infection in utero; and determining whether pregnant women act as a reservoir for malaria.

Without wishing to be limited by a closed list, p5 and p8 were selected because: First, antibody responses were highly increased at delivery in women that experienced a detected infection in agreement with the short time to double the antibody levels estimated in relation to other antigens. Second, antibodies did not increased with increasing parity of the women in accordance with a half-life and time to sero-negativisation below the average time reported in Mozambique for a second pregnancy to occur. Third, the seroprevalence at delivery was not below but similar with the prevalence of infection detected during that particular pregnancy.

Immunoreactive and exposure-dependent new VAR2CSA peptides with antibody responses able to provide information about malaria changes over time and space were identified. Furthermore, peptides that were suitable to confirm zero incidence in pregnant women attending antenatal clinics as sentinel for malaria surveillance in surrounding community were identified. Moreover, the value of VAR2CSA-based serology to detect recent reductions in exposure to P. falciparum associated with the use of Intermittent Preventive Treatment with different antimalarials was also assessed.

According to at least some embodiments, antibody levels may optionally be measured in a subject in a number of different ways, including but not limited to, bead-based assays (e.g. AlphaScreen® or Luminex® technology), the enzyme linked immuosorbent assay (ELISA), protein microarrays and the luminescence immunoprecipitation system (LIPS). All the aforementioned methods generate a continuous measurement of antibody.

### BRIEF DESCRIPTION OF THE DRAWINGS

FIG. 1A-1 shows the study profile;

FIGS. 1A-2 to 1B show the immunoreactivity and exposure-dependence of VAR2CSA peptides. A) The dot plot shows the normalized median fluorescence intensity (nMFIs) measured in Mozambican pregnant women against the protein array and the peptide array. Dots represent nMFI of each pregnant woman, red lines correspond to geometric mean and T-bars represent the 95% CI. Red dashed line represents the mean nMFI from bovine serum albumin (BSA) plus 3 standard deviations (BSA reactivity threshold). B) Ratio of nMFIs measured in pregnant Mozambican women and pregnant Spanish women (black circle). Bars represent the seroprevalence from pregnant Spanish women. T-bars correspond to the 95% confidence interval (CI). Red dashed line represents the 5% seroprevalence threshold.

FIG. 2A shows VAR2CSA peptides able to mirror malaria trends in Mozambique. Ratio of normalized median fluorescence intensity (nMFIs) measured in pregnant Mozambican women with samples collected during 2010 compared with samples collected during 2003-2005 corresponding to a trend of malaria decrease (bottom of the graph). Ratio of nMFIs measured in pregnant Mozambican women with samples collected during 2011-2012 were compared with samples collected during 2010 corresponding to a trend of increase. Antibodies were measured against the recombinant proteins and peptides. All regressions were adjusted by treatment, parity, age and HIV. T bars correspond to 95% CI, Red color means p-value >0.1.

FIG. 3. VAR2CSA peptides eliciting IgG responses rapidly generated with a limited life-time. A) Ratio of normalized median fluorescence intensity (nMFIs) measured at delivery in pregnant Mozambican women with at least one infection detected during pregnancy (n=49) compared with Mozambican pregnant women without detected infection (n=160). Women were considered infected during pregnancy if peripheral or placental blood samples were positive by microscopy or qPCR at any time-point of collection, if P. falciparum detected by hospital passive case detection (PCD), or if histology positive (active, chronic or past) on the sub-set of women from longitudinal cohort. B) Ratio of nMFIs measured at delivery in pregnant Mozambican women with different parasite density (below or above 200). C) Time to double the antibody levels (T2x). D) Half-life of antibody levels. E) Time to sero-negativisation (TSN) of antibodies. F) Ratio of nMFIs measured at delivery in multigravidae Mozambican women (n=175) compared with primigravidae Mozambican women (n=64). E) Seroprevalence at delivery (bars) measured in the 239 pregnant Mozambican women. Red dashed line shows the total prevalence on infection detected during pregnancy and the green line the prevalence only detected at delivery. All regressions were adjusted by treatment, parity, age and HIV. T2x, half-life and TSN was analyzed using log-linear mixed-effects regression models incorporating Gaussian random intercepts and results were expressed as time in weeks. T bars correspond to 95% CI.

FIG. 3H shows antibody dynamics during pregnancy.

FIGS. 4A-D shows (sero)prevalence of Plasmodium falciparum among Mozambican pregnant women at delivery, according to year. Panels A, B and C show the seroprevalence against VAR2CSA peptides, recombinant proteins and non-VAR2CSA antigens, respectively. Antibodies were measured by multiplex bead-based immunoassay in plasma samples collected at delivery (n=67 for 2004, n=81 for 2005, n=177 for 2010, n=244 for 2011 and n=272 for 2012) and seroprevalences were obtained by finite mixture models (FMM) for VAR2CSA-based antigens or by negative controls means for non-VAR2CSA antigens. Panel D shows the prevalence of infection measured by quantitative polymerase chain reaction (qPCR) in both peripheral and placental blood collected at delivery. P values are based on a multivariate analysis adjusted for human immunodeficiency virus (HIV) status, parity, age and treatment. T bars represent 95% confidence intervals.

FIG. 4E shows division of seroprevalences by qPCR prevalences in Benin (high transmission) and Mozambique (low transmission).

FIG. 5: (Sero)Prevalences of Plasmodium falciparum among pregnant women, according to country. Panel A, B and C shows the seroprevalence against VAR2CSA peptides, recombinant proteins and non-VAR2CSA antigens, respectively. Antibodies were measured by multiplex bead-based immunoassay in plasma samples collected at delivery (HIV negative: n=854 for Benin, n=131 for Gabon, n=485 for Mozambique and n=31 for Tanzania; HIV-infected: n=362 for Mozambique and n=296 for Kenya) and seroprevalences were obtained by finite mixture models (FMM) for VAR2CSA-based antigens or by negative controls means for non-VAR2CSA antigens. Panel D shows the prevalence of infection measured by quantitative polymerase chain reaction (qPCR) in both peripheral and placental blood collected at delivery (HIV negative: n=307 for Benin, n=89 for Gabon, n=332 for Mozambique and n=31 for Tanzania; HIV-infected: n=322 for Mozambique and n=273 for Kenya). P values are based on a multivariate analysis adjusted for parity, age and treatment. T bars represent 95% confidence intervals.

FIG. 6 shows (sero)prevalences of Plasmodium falciparum among pregnant women, according to intermittent preventive treatment intervention group. Panels A, B and C show the seroprevalence against VAR2CSA peptides, recombinant proteins and non-VAR2CSA antigens, respectively. Antibodies were measured by multiplex bead-based immunoassay in plasma samples collected at delivery (HIV negative: n=996 for mefloquine [MQ], n=505 for sulfadoxine-pyrimethamine [SP]; HIV-infected: n=317 for MQ and n=342 for Placebo) and seroprevalences were obtained by finite mixture models (FMM) for VAR2CSA-based antigens or by negative controls means for non-VAR2CSA antigens. Panel D shows the prevalence of infection measured by quantitative polymerase chain reaction (qPCR) in both peripheral and placental blood collected at delivery (HIV negative: n=490 for MQ, n=262 for SP; HIV-infected: n=283 for MQ and n=313 for Placebo). P values are based on a multivariate analysis adjusted for parity and age. T bars represent 95% confidence intervals.

FIG. 7 shows (sero)prevalences of Plasmodium falciparum among pregnant women, according to anemia status. Panels A, B and C show the seroprevalence against VAR2CSA peptides, recombinant proteins and non-VAR2CSA antigens, respectively. Antibodies were measured by multiplex bead-based immunoassay in plasma samples collected at delivery (HIV negative: n=571 for anemic [A], n=889 for non-anemic [non-A]; HIV-infected: n=242 for anemic and n=417 for non-anemic) and seroprevalences were obtained by finite mixture models (FMM) for VAR2CSA-based antigens or by negative controls means for non-VAR2CSA antigens. Panel D shows the prevalence of infection measured by quantitative polymerase chain reaction (qPCR) in both peripheral and placental blood collected at delivery (HIV negative: n=289 for anemic [A], n=463 for non-anemic [non-A]; HIV-infected: n=214 for anemic and n=378 for non-anemic). P values are based on a multivariate analysis adjusted for parity, age and treatment. T bars represent 95% confidence intervals.

FIG. 8 shows 3D models of DBL1X-ID1 showing selected peptides. (A) Ribbon representation of DBL1X-ID1 showing p5 colored in blue and p8 in orange. (B) Space-feeling representation of DBL1X-ID1 showing p5 colored in blue and p8 in orange. (C) Space-feeling representation of DBL1X-ID1 showing p5 epitopos (p5E) colored in blue and p8 epitopos (p8E) in Orange predicted by BepiPred.

### DESCRIPTION OF AT LEAST SOME EMBODIMENTS

The present invention, in at least some embodiments, is of a system, method, apparatus and diagnostic test for monitoring infections by Plasmodium falciparum that is specific for pregnant women. The monitoring is performed by examining samples from the pregnant women, typically blood samples, for the presence of antibodies to a known P. falciparum protein, VAR2CSA. Preferably, the antibodies bind specifically to p5 and/or p8.

Detection of pregnancy-specific antibodies against VAR2CSA (the parasite antigen used by P. falciparum malaria parasites to sequester in the placenta [1]) in malaria-exposed pregnant women can inform about recent infections (during pregnancy). So, it can be used for:

1. To estimate the level of malaria burden/transmission: the presence of antibodies indicates that the woman was infected during pregnancy (serology is a historical record of infection). The advantage of VAR2CSA-serology, compared to other serological approaches in the general population [2, 3], is that it allows monitoring recent changes (during one pregnancy) in malaria burden. Measuring antibodies against VAR2CSA can be more powerful to detect circulating parasites than detecting active infections (i.e., the parasite itself) when the prevalence is low due to substantial drops in malaria incidence [4]. So, this tool can be very useful for surveillance in malaria elimination activities.

2. Proxy for parasite prevalence in the community: Malaria estimates in pregnant women using this serology could be used as an indicator of how much malaria is in the general population. The relatively easy access of pregnant women at antenatal clinics would reduce surveillance costs compared to logistically complex cross-sectionals in the community, increasing long-term sustainability when malaria transmission has decreased to a point in which it ceases to be a public health concern and efforts become more relaxed [5].

3. To monitor the absence of malaria transmission resulting from elimination activities: Demonstrating freedom from infection [6] requires a high sample size to conclude that there are no parasites in the area. Given their high risk of malaria infection [4], targeted sampling of pregnant women through this serological approach would increase the probability of detecting infections if present as well as the confidence to confirm absence of infection (risk-based surveillance). A negative result by VAR2CSA-serology (i.e., no antibodies detected) in pregnant women can be used as a signal of malaria elimination (free of circulating parasites).

4. To assess if pregnant women are reservoirs of malaria transmission: Community chemotherapy campaigns to reduce malaria transmission often exclude pregnant women due to safety concerns related to the antimalarial, especially during first trimester. For this reason, the use of this serology can inform about the existence of these reservoirs in pregnant women.

5. To detect reintroduction of malaria in settings targeted by elimination efforts: Increases in antibody levels against VAR2CSA in pregnant women would indicate potential resurgences of malaria and thus be used to guide timely approaches to control increases in transmission.

6. To assess the impact of control, preventive and elimination tools: Reductions in antibody responses against VAR2CSA can be indicative of lower parasite burden and thus suggest that intervention packages are working well. On the contrary, increases in antibody responses could be used as an early warning signal that the control tools for malaria are not optimal.

7. To identify localized geographical areas with higher burdens of malaria (hotspots): Pregnant women with a positive results with the VAR2CSA-serological test could be indicative of malaria transmission in their area of residence and then be used to target efforts for malaria control elimination in these areas.

8. To identify pregnancies at risk: It has been described a larger impact of infections at the beginning of pregnancy (when the fetus is developing) than at delivery [7-9]. A positive VAR2CSA-serology could be used as an indication of early infection during pregnancy and thus indicate a higher risk of low birth weight or prematurity.

9. To guide design of pregnancy-specific vaccines against malaria: As VAR2CSA is a potential target for vaccine development [10], the serological test can be used to guide successful immunization with the vaccine (if it gets to the point of be used as a public health tool) and help to understand the basis of immune protection during pregnancy.

### REFERENCES

Salanti, A., et al., Selective upregulation of a single distinctly structured var gene in chondroitin sulphate A-adhering Plasmodium falciparum involved in pregnancy-associated malaria. Mol Microbiol, 2003. 49(1): p. 179-91.
2. Drakeley, C. and J. Cook, Chapter 5. Potential contribution of sero-epidemiological analysis for monitoring malaria control and elimination: historical and current perspectives. Adv Parasitol, 2009. 69: p. 299-352.
3. Corran, P., et al., Serology: a robust indicator of malaria transmission intensity? Trends Parasitol, 2007. 23(12): p. 575-82.
4. Ataide, R., A. Mayor, and S. J. Rogerson, Malaria, primigravidae, and antibodies: knowledge gained and future perspectives. Trends Parasitol, 2014. 30(2): p. 85-94.
5. Cohen, J. M., et al., Malaria resurgence: a systematic review and assessment of its causes. Malar J, 2012. 11: p. 122.
6. Stresman, G., A. Cameron, and C. Drakeley, Freedom from Infection: Confirming Interruption of Malaria Transmission. Trends Parasitol, 2017.
7. Cottrell, G., et al., The importance of the period of malarial infection during pregnancy on birth weight in tropical Africa. Am J Trop Med Hyg, 2007. 76(5): p. 849-54.
8. Huynh, B. T., et al., Influence of the timing of malaria infection during pregnancy on birth weight and on maternal anemia in Benin. Am J Trop Med Hyg, 2011. 85(2): p. 214-20.
9. Valea, I., et al., An analysis of timing and frequency of malaria infection during pregnancy in relation to the risk of low birth weight, anaemia and perinatal mortality in Burkina Faso. Malar J, 2012. 11: p. 71.
10. Pehrson, C., et al., Pre-clinical and clinical development of the first placental malaria vaccine. Expert Rev Vaccines, 2017. 16(6): p. 613-624.

### Example 1—Antibody Selection

Methods

Ethics Statement

The study was approved by the Ethics Committees from the Hospital Clinic of Barcelona (Spain), the Comite Consultatif de Déontologie et d'Ethique from the Institut de Recherche pour le Développement (France), the Centers for Disease Control and Prevention (USA), and National Ethics Review committees from each malaria endemic country participating in the study. Written informed consent was obtained from all the participants.

Study Sites and Population

The women included in this study were recruited during 2 clinical trials of intermittent preventive treatment during pregnancy (IPTp) between 2003-2005 (Clinical trials.gov NCT00209781) [17] in Mozambique and between 2010-2012 (NCT00811421) [18,19] in Mozambique but also Benin, Gabon, Kenya and Tanzania. Women recruited between 2003-2005 received two doses of sulfadoxine-pyrimethamine (SP) [17] and women recruited between 2010-2012 received two doses of mefloquine (MQ) or SP, if the women were HIV-negative [19] or three doses of MQ or placebo, if they were HIV-positive receiving trimethoprim-sulfamethoxazole prophylaxis [18]. All women included in the study received bed nets treated with long-lasting insecticide. At delivery, HIV serostatus was assessed using a rapid diagnostic test and hemoglobin was determined in capillary blood sample using mobile devices (HemoCue, Hemocontrol and Sysmex KX analyzer). Tissue samples from the maternal side of the placenta, as well as maternal peripheral-, placental-blood samples were collected at delivery. Dried blood spots onto filter paper were prepared (50 ul) and blood was collected into EDTA vacutainers and centrifuged, with the plasma stored at −20° C. More than one peripheral blood sample was collected during pregnancy from a longitudinal cohort composed by part of the Mozambican women recruited during 2011-2012 [18,19]. Clinical malaria episodes were treated according to national guidelines at the time of the study. Biases due to pooling of data from these two clinical trials were minimized by the use of similar protocols and procedures during the two trials. Finally, as controls, 49 plasma samples were collected from pregnant women, recruited at delivery at the Hospital Clinic in Barcelona during 2010.

In Mozambique, the prevalence of malaria infection among pregnant women highly decreased from 2003 to 2010 and then slight increased until 2012 [20]. The estimated proportion of 2-10 years old children with P. falciparum infection (PfPR₂₋₁₀), derived from the Malaria Atlas Project geostatistical prediction model [21] was 29% in 2003-2005, 5% in 2010 and 9% in 2011-2012, in agreement with previous measures. Similar prevalences were obtained for clinical malaria cases reported from 2003 to 2012 in children less than five years of age observed at the Manhiça District Hospital (32% in 2003-2005, 8% in 2010 and 14% in 2011-2012, unpublished data).

Antigens

Recombinant proteins used were VAR2CSA Duffy binding-like domains (DBL3X, DBL5ϵ and DBL6ϵ, from 3D7 strain) [11,22], apical membrane antigen 1 (AMA1, from 3D7 strain) [23], merozoite surface protein-1, 19-kDa, (MSP1₁₉, from 3D7 strain) [24], all produced at ICGEB (New Delhi, India). Clostridium tetani, tetanus toxin, purchased from Santa Cruz Biotechnology (Dallas, Tex.). We designed 25 synthetic peptides covering conserved and semi conserved regions from VAR2CSA [25]. A circumsporozoite peptide (pCSP) of 64 aminoacids (NVDP[NANP]₁₅) was also included [26]. Peptides were synthetized by G1 Biochem (Xangai, China) and median purity was estimated as 79% (range: 71-91%) by HPLC and mass spectrometry.

Parasitological Determinations

Thick and thin blood films, as well as placental biopsies, were read for Plasmodium species detection according to standard, quality-controlled procedures [27-29]. Blood onto filter papers were tested for the presence and density of P. falciparum in duplicate by means of a real-time quantitative polymerase chain-reaction (qPCR) assay targeting 18S ribosomal RNA (rRNA) [30,31]. Past placental infection was defined by the presence of malaria pigment (i.e., hemozoin) without parasite detection on placental histologic examination, and chronic placental infection was defined by the presence of malaria pigment in combination with the detection of parasites [20]. P. falciparum infection at delivery was defined if peripheral or placental blood samples were positive by microscopy or qPCR or if histology positive (active or chronic). Infection during pregnancy was defined if peripheral or placental blood samples were positive by microscopy or qPCR at any time-point of collection, if P. falciparum detected by hospital passive case detection (PCD), or if histology positive (active, chronic or past) on the sub-set of women from longitudinal cohort.

Bead-Based Immunoassay

Two multiplex suspension array panels were constructed to quantify IgG responses against P. falciparum recombinant proteins and synthetic peptides, using the xMAP™ technology and the Luminex® 100/200™ System (Luminex® Corp., Austin, Tex.). MagPlex® microspheres (magnetic carboxylated polystyrene microparticles, 5.6 μm) with different spectral signatures were selected for each protein (DBL3X, DBL5ϵ, DBL6ϵ, AMA1 and MSP1₁₉), peptides (25 VAR2CSA peptides and pCSP), tetanus toxin and bovine serum albumin (BSA). Antigens were covalently coupled to beads following a modification of the Luminex® Corporation protocol [25]. Protein and peptide multiplex arrays were prepared by pooling together equal volumes of coated beads. Plasma samples or the product of DBS elution were analyzed in duplicate at dilution 1:400 for the protein array and 1:100 for the peptide array. A hyperimmune plasma pool composed by 23 plasmas from malaria infected Mozambican pregnant women (HIP-VAR2CSA) was included in each assay plate, in addition to blanks (wells without sample) to assess background levels. A minimum of 50 microspheres were read per spectral signature and results were exported as crude median fluorescent intensity (HFI). Duplicates were averaged and background MFIs were subtracted. A total of 224 plates were analyzed and the intra-assay variation (mean CV of replicates from 20 plasma samples per plate) ranged from 1.4% to 7.3% for the protein array and from 2.5% to 12.4% for the peptide array. The inter-assay variation (variability of positive pool [HIP-VAR2CSA] between 224 plates) was 5% for the protein array and 26% for the peptide array. Results were normalized (nMFI) to account for plate-to-plate variation by multiplying the background subtracted MFI of each sample with the value of the positive pool in the same plate and dividing by the median of positive pools in all plates.

Reconstitution of Blood Drops onto Filter Paper

Antibodies were eluted from DBS from Gabon, Tanzania and Kenya, as previously described [25,32]. Briefly, to achieve a concentration of eluted blood proteins equivalent to a 1:50 plasma dilution antibodies were eluted from four spots of approximately 3 mm in diameter with 200 μl Luminex® assay buffer (1% BSA, 0.05% sodium azide in filtrated PBS [Phosphate-buffered saline]). Appropriate elution was considered based on visual inspection (white spots against a reddish background) of spot reconstitution, adequate hemoglobin levels (above the highest quartile [7.4 mg/l] of samples with inappropriate visual aspect) measured by spectrophotometry and high anti-tetanus toxin nMFIs measured in the eluted product (above the lowest quartile [11563,5 nMFI]) by Luminex® [25]. Controls blood drops were artificially prepared using fresh erythrocytes (blood type 0, assuming a hematocrit approximately of 50%) and freeze plasma from 49 Spanish pregnant women. Filter papers were stored avoiding humidity at −20° C. in Barcelona.

Protein 3D Models

The 3D-structure of DBL1X-ID1 was calculated by submitting the 3D7 sequence (with domain limits defined by [33]) to the HHPred server (http://toolkit.tuebingen.mpg.de/hhpred). The structure with highest HHPred score, corresponding to the DBLlalfa domain of the VarO strain (Protein Data Bank [PDB] 2yk0 [34]), was selected for homology modeling in MODELLER based on the default alignment. Molecular graphics were generated in UCSF Chimera version 1.5.3 [35].

### Definitions and Statistical Analysis

Women were classified as primigravid (first pregnancy) and multigravid (at least one previous pregnancy). Age was categorized as younger than 20 years, 20 to 24 years, or 25 years of age or older [11]. Anemia was defined by hemoglobin level at delivery below 11 mg/l [36].

Presence or absence of antibodies was defined by finite mixture models (FMM) for pregnancy-specific antigens (VAR2CSA peptides and recombinant domains) [25,32] and by the mean plus 3 standard deviation (SD) of IgG response from pregnant women from Barcelona for general malaria antigens (AMA1, MSP1₁₉, and pCSP). Immunoreactivity was verified if nMFI mean above BSA recognition (mean BSA+3 SD).

Intra-assay variation was calculated as the mean coefficient of variation (CV=SD/Mean*100%) from replicates analyzed in each plate. The inter-assay variation was calculated as the CV of the median MFI from all antigens included in each multiplex array measured in the positive pool repeated in all plates, before normalization.

Data was fitted to a normal distribution by logarithmic transformation of nMFIs. Participant's baseline characteristics and parasitological outcomes were compared between study times and areas by univariate analysis (Fisher's tests for binary outcomes or t-Student test for continuous outcomes). Responses were considered not restricted to malaria when seroprevalences were 5% or high among Spanish pregnant women.

Linear regression models were used to compare antibody levels from Mozambican and Spanish pregnant women. The capacity of antibody levels to mimic malaria trends in Mozambique was analyzed by linear regression models (2010 was compared with 2003-2005 corresponding to a trend of malaria decrease and 2011-2012 with 2010 to a trend of increase) adjusted by treatment, parity, age and HIV.

The impact of P. falciparum infection during pregnancy on antibodies at delivery was assessed in linear regression models adjusted by age, parity, HIV and treatment. Changes in antibody levels due to parasite density (below or above 200) were assessed by linear regression models adjusted by age, parity, HIV and treatment.

The adjusted effect of infection on antibody levels was analyzed using log-linear mixed-effects regression models incorporating Gaussian random intercepts. This resulted in an estimate of the rates of antibody dynamics (increase or decay), assuming a single exponential model. Time to double the antibody levels (T2x) and half-lives were calculated in weeks from the estimated rates and the boundaries at 95% confidence interval obtained from mixed-effects models for subjects suffering a P. falciparum infection at follow-up independent of antibody status at recruitment (Ab[−or+]/Pf+) and subjects seropositive at recruitment and no infection detected on follow-up (Ab+/Pf−), respectively [37,38]. In situations that the increase rate is a negative value (rate below 1) or the decay rate is a positive value (rate above 1), the calculated T2x or half-life was reported as infinity. Similarly, the time to sero-reversion was calculated for these subjects using the ratio of the seropositivity cutoff by the average antibody titers at recruitment as decay, i.e. how many times the average titers need to be reduced to be equal the cutoff.

The impact of parity (multigravid vs primigravid) on antibody levels at delivery was verified by linear regression models adjusted by age, HIV and treatment.

Seroprevalences between countries (Benin, Gabon and Mozambique for HIV-uninfected and Kenya and Mozambique for HIV-infected), between IPTp intervention group (MQ vs SP for HIV-uninfected and MQ vs Placebo for HIV infected) and anemia status were compared by logistic regression models adjusted by parity, age and treatment.

The modification of the associations by HIV infection or parity was assessed by including interaction terms into the regression models.

Statistical analyses were performed with Stata/SE software (version 12.0; StataCorp) and Graphpad Prism (version 6, Graphpad, Inc). P-values of less than 0.05 were considered to indicate statistical significance.

Results

1. Sample Size and Description

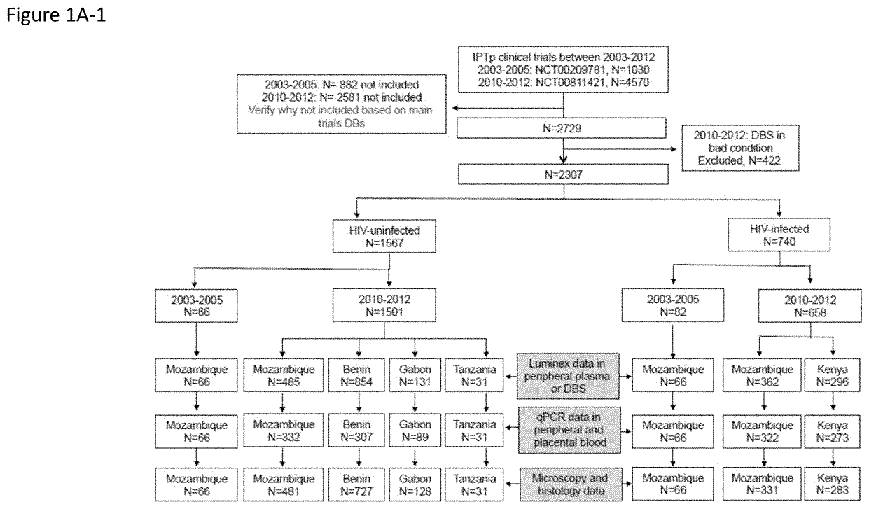

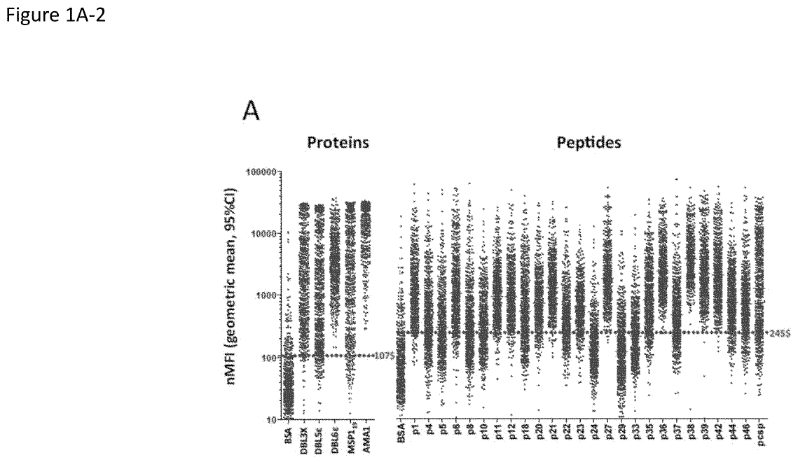

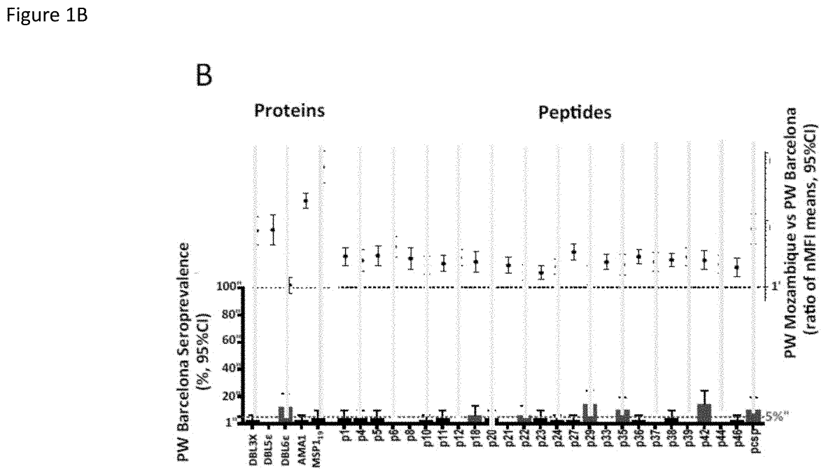

Antibodies were measured in 2729 samples (1849 plasmas and 880 DBS) from pregnant women collected at delivery in the context of two clinical trials of intermittent preventive treatment of malaria in pregnancy from 2003 to 2012 (FIG. 1A-1). Table 1A shows some characteristics of the women included in the analysis.

TABLE 1A
Women included in the analysis (samples description by HIV status and trial)

| | HIV uninfected | | | | | HIV infected | | |
|---|---|---|---|---|---|---|---|---|
| | 2003-2005 | 2010-2012 | | | | 2003-2005 | 2010-2012 | |
| | Mozambique | Mozambique*ψ | Benin* | Gabon* | Tanzania | Mozambique | Mozambique**ψ | Kenya |
| Variable | N = 66 | N = 485 | N = 854 | N = 131 | N = 31 | n = 82 | n = 362 | N = 296 |
| Parity, n(%) | | | | | | | | |
| PG | 17 (26) | 181 (37) | 188 (22) | 38 (29) | 16 (52) | 28 (34) | 46 (13) | 22 (7) |
| MG | 49 (74) | 304 (63) | 666 (78) | 93 (71) | 15 (48) | 54 (66) | 318 (87) | 274 (93) |
| Age, n(%) | | | | | | | | |
| 15-20 | 19 (29) | 181 (37) | 86 (10) | 42 (32) | 5 (16) | 27 (33) | 41 (11) | 15 (5) |
| 20-25 | 17 (26) | 123 (26) | 281 (33) | 45 (34) | 14 (45) | 27 (33) | 84 (23) | 96 (32) |
| >25 | 30 (45) | 181 (37) | 487 (57) | 44 (34) | 12 (39) | 28 (34) | 237 (66) | 185 (63) |
| IPTp, n(%) | | | | | | | | |
| SP | 66 (100) | 151 (31) | 288 (34) | 55 (42) | 11 (36) | 82 (100) | 0 (0) | 0 (0) |
| MQ | 0 (0) | 334 (69) | 566 (66) | 76 (58) | 20 (64) | 0 (0) | 178 (49) | 139 (47) |
| Placebo | 0 (0) | 0 (0) | 0 (0) | 0 (0) | 0 (0) | 0 (0) | 184 (51) | 157 (53) |
| Year delivery, n(%) | | | | | | | | |
| 2003/4 | 22 | 0 (0) | 0 (0) | 0 (0) | 0 (0) | 45 (55) | 0 (0) | 0 (0) |
| 2005 | 44 | 0 (0) | 0 (0) | 0 (0) | 0 (0) | 37 (45) | 0 (0) | 0 (0) |
| 2010 | 0 (0) | 99 (21) | 457 (54) | 22 (17) | 4 (13) | 0 (0) | 100 (28) | 57 (19) |
| 2011 | 0 (0) | 293 (60) | 335 (39) | 60 (46) | 27 (87) | 0 (0) | 188 (52) | 144 (49) |
| 2012 | 0 (0) | 93 (19) | 62 (7) | 49 (37) | 0 (0) | 0 (0) | 74 (20) | 95 (32) |
| Malaria status, n(%)# | | | | | | | | |
| positive | 36 (55) | 32 (10) | 201 (54) | 10 (12) | 1 (3) | 52 (63) | 10 (3) | 27 (10) |
| negative | 30 (45) | 302 (90) | 170 (46) | 76 (88) | 30 (97) | 30 (37) | 303 (97) | 244 (90) |
| qPCR, n(%)$ | | | | | | | | |
| positive | 17 (26) | 27 (8) | 127 (41) | 9 (10) | 0 (0) | 21 (26) | 9 (3) | 22 (8) |
| negative | 49 (74) | 305 (92) | 180 (59) | 80 (90) | 31 (100) | 61 (74) | 313 (97) | 251 (92) |
| Microscopy, n(%)$ | | | | | | | | |
| positive | 35 (53) | 13 (3) | 110 (15) | 3 (2) | 1 (3) | 44 (54) | 8 (2) | 15 (5) |
| negative | 31 (47) | 468 (97) | 616 (85) | 125 (98) | 30 (97) | 38 (46) | 323 (98) | 268 (95) |
| Placental histology, n(%) | | | | | | | | |
| acute | 9 (14) | 1 (0) | 12 (2) | 2 (2) | 1 (3) | 7 (8) | 5 (2) | 5 (2) |
| chronic | 0 (0) | 3 (1) | 66 (9) | 1 (1) | 0 (0) | 0 (0) | 1 (0) | 4 (1) |
| past | 26 (39) | 18 (4) | 75 (10) | 11 (9) | 0 (0) | 36 (44) | 14 (4) | 44 (16) |
| negative | 31 (47) | 459 (95) | 574 (79) | 114 (89) | 30 (97) | 39 (48) | 311 (94) | 230 (81) |

PG, primigravidae;
MG, multigravidae;
SP, sulfadoxine-pyrimethamine;
MQ, mefloquine;
IPTp, intermitent preventive treatment during pregnancy;
qPCR, quantitative polimerase chain reaction;
#Positive by microscopy or qPCR or histology (active or chronic).
$qPCR and microscopy assessed in peripheral and placental blood
*HIV-uninfected missing data: Mozambique (153 qPCR, 4 microscopy and histology); Benin (547 qPCR, 127 microscopy and histology); Gabon(42 qPCR, 3 microscopy and histology)
**HIV-infected missing data: Mozambique (40 qPCR, 31 microscopy and histology); Kenya (23 qPCR, 13 microscopy and histology)
ψ40% of HIV-uninfected and 12% of HIV-infected were followed during pregnacy

Abbreviations are as follows: PG, primigravidae; MG, multigravidae; SP, sulfadoxine-pyrimethamine; MQ, mefloquine; IPTp, intermittent preventive treatment during pregnancy; qPCR, quantitative polymerase chain reaction; # Positive by microscopy or qPCR or histology (active or chronic).

$ qPCR and microscopy assessed in peripheral and placental blood

*HIV-uninfected missing data: Mozambique (153 qPCR, 4 microscopy and histology); Benin (547 qPCR, 127 microscopy and histology); Gabon (42 qPCR, 3 microscopy and histology)

**HIV-infected missing data: Mozambique (40 qPCR, 31 microscopy and histology); Kenya (23 qPCR, 13 microscopy and histology)

Ψ 40% of HIV-uninfected and 12% of HIV-infected were followed during pregnancy

Results from 422 DBS samples were excluded because of inappropriate elution of antibodies. From the total of 2307 pregnant women finally included for analysis (Table 1), 1567 (68%) were HIV-negative and 740 (32%) were HIV-infected. Among samples from HIV-negative women, 854 (55%) were plasmas from Benin, 551 (35%) were plasmas from Mozambique, 131 (8%) were DBS from Gabon and 31 (2%) were DBS from Tanzania, whereas 444 (60%) plasmas and 296 (40%) DBS were from HIV-infected Mozambican and Kenyan women, respectively. Among the Mozambican women 148 (55% HIV infected) were from a trial that occurred between 2003 and 2005 and 847 (43% HIV infected) were from a second trial that occurred between 2010 and 2012, together with all women included in the study from Benin, Gabon, Tanzania and Kenya. A total of 239 pregnant Mozambican women participating on the second trial were followed during pregnancy and 2 plasma samples were collected during pregnancy and 1 at delivery (total of 696 plasmas analyzed; exception of 21 women that only 1 plasma samples was collected during pregnancy plus delivery). The women included in this study were similar in terms of baseline characteristics with all 5600 women participating in the randomized trials (shown in Table 1B).

TABLE 1B
Characteristics of the women participating in the intermittent
preventive treatment trials and those not included in the study

| | HIV uninfected | | | | | | HIV infected | | | | | |
|---|---|---|---|---|---|---|---|---|---|---|---|---|
| | 2003-2005 | | | 2010-2012* | | | 2003-2005 | | | 2010-2012** | | |
| | included | | | included | | | included | | | included | | |
| | yes | no | | yes | no | | yes | no | | yes | no | |
| Variable | N = 66 | N= | p | N = 1501 | N= | p | N = 82 | N= | p | N = 658 | N= | p |
| Parity, n(%) | | | | | | | | | | | | |
| PG | 17 (26) | | | 423 | | | 28 (34) | | | 68 | | |
| MG | 49 (74) | | | 1078 | | | 54 (66) | | | 592 | | |
| Age, n(%) | | | | | | | | | | | | |
| 15-20 | 19 (29) | | | 314 | | | 27 (33) | | | 56 | | |
| 20-25 | 17 (26) | | | 463 | | | 27 (33) | | | 180 | | |
| >25 | 30 (45) | | | 724 | | | 28 (34) | | | 422 | | |
| IPTp, n(%) | | | | | | | | | | | | |
| SP | 66 (100) | | | 505 | | | 82 (100) | | | 0 (0) | | |
| MQ | 0 (0) | | | 996 | | | 0 (0) | | | 317 | | |
| Placebo | 0 (0) | | | 0 (0) | | | 0 (0) | | | 341 | | |
| Year delivery, n(%) | | | | | | | | | | | | |
| 2003/4 | 22 | | | 0 (0) | | | 45 (55) | | | 0 (0) | | |
| 2005 | 44 | | | 0 (0) | | | 37 (45) | | | 0 (0) | | |
| 2010 | 0 (0) | | | 582 | | | 0 (0) | | | 157 | | |
| 2011 | 0 (0) | | | 715 | | | 0 (0) | | | 332 | | |
| 2012 | 0 (0) | | | 204 | | | 0 (0) | | | 169 | | |
| Malaria status, n(%)# | | | | | | | | | | | | |
| positive | 36 (55) | | | 244 | | | 52 (63) | | | 37 | | |
| negative | 30 (45) | | | 578 | | | 30 (37) | | | 547 | | |
| qPCR, n(%)$ | | | | | | | | | | | | |
| positive | 17 (26) | | | 163 | | | 21 (26) | | | 31 | | |
| negative | 49 (74) | | | 596 | | | 61 (74) | | | 864 | | |
| Microscopy, n(%)$ | | | | | | | | | | | | |
| positive | 35 (53) | | | 127 | | | 44 (54) | | | 23 | | |
| negative | 31 (47) | | | 1239 | | | 38 (46) | | | 591 | | |
| Placental histology, n(%) | | | | | | | | | | | | |
| acute | 9 (14) | | | 16 | | | 7 (8) | | | 10 | | |
| chronic | 0 (0) | | | 70 | | | 0 (0) | | | 5 | | |
| past | 26 (39) | | | 104 | | | 36 (44) | | | 58 | | |
| negative | 31 (47) | | | 1177 | | | 39 (48) | | | 541 | | |

PG, primigravidae;
MG, multigravidae;
SP, sulfadoxine-pyrimethamine;
MQ, mefloquine;
IPTp, intermitent preventive treatment during pregnancy;
qPCR, quantitative polimerase chain reaction;
#Positive by microscopy or qPCR or histology (active or chronic).
$qPCR and microscopy assessed in peripheral and placental blood
*HIV-uninfected missing data: 742 qPCR, 134 microscopy and histology
**HIV-infected missing date: 63 qPCR, 44 microscopy and histology

Abbreviations are as follows: PG, primigravidae; MG, multigravidae; SP, sulfadoxine-pyrimethamine; MQ, mefloquine; IPTp, intermittent preventive treatment during pregnancy; qPCR, quantitative polymerase chain reaction;

# Positive by microscopy or qPCR or histology (active or chronic).

$ qPCR and microscopy assessed in peripheral and placental blood

*HIV-uninfected missing data: 742 qPCR, 134 microscopy and histology

**HIV-infected missing data: 63 qPCR, 44 microscopy and histology

2. Selection of VAR2CSA Peptides Recognized by Antibodies from Malaria Exposed Pregnant Women and Responses Able to Mirror Malaria Trends in Mozambique Between 2003 and 2012

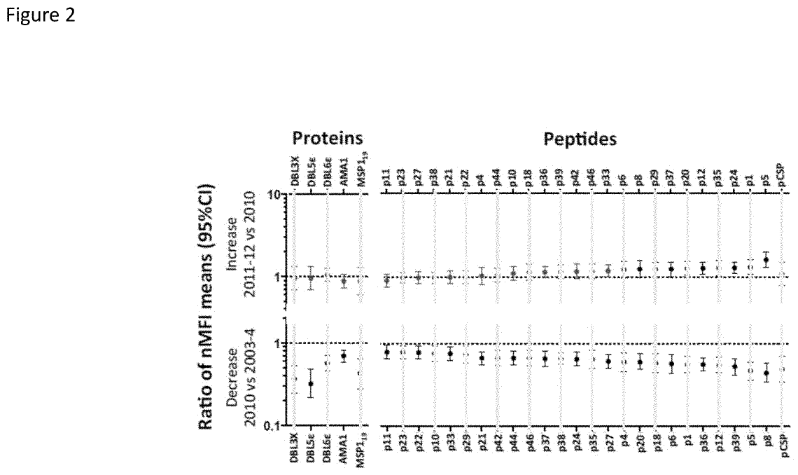

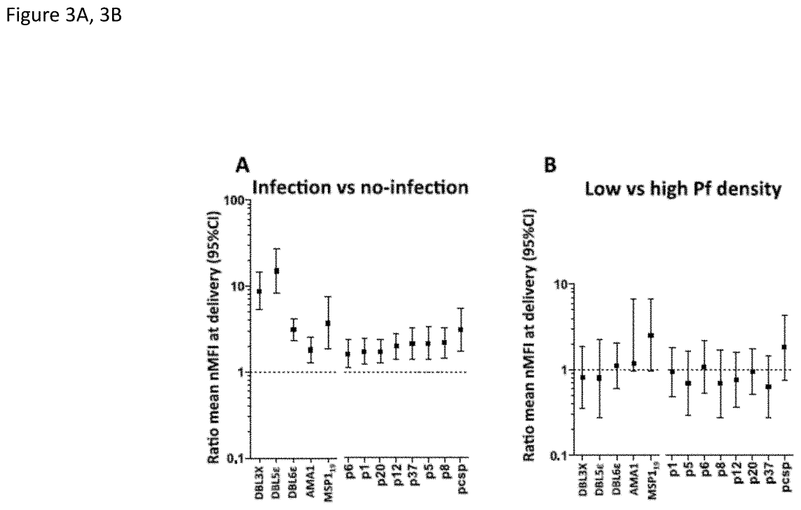

IgGs from 641 pregnant Mozambican women delivering between 2003 and 2012 recognized 22/25 VAR2CSA peptides (exception: p24, p29 and p33), all VAR2CSA recombinant proteins (DBL3X, DBL5ϵ, DBL6ϵ) and all non-VAR2CSA P. falciparum antigens (AMA1, MSP1₁₉ and pCSP) at levels above BSA recognition (mean nMFI from each malaria antigen above mean nMFI from BSA plus 3 SD) (FIG. 1A-2, Table 2A).

Table 2A shows a selection of VAR2CSA peptides recognized by antibodies from malaria exposed pregnant women and responses able to mirror malaria trends in Mozambique between 2003 and 2012.

TABLE 2A
Selection of VAR2CSA peptides

| | Exposure dependence | | | | | | |
|---|---|---|---|---|---|---|---|
| | | | SeroPrev | Decrease | | Increase | |
| | PW Mz vs PW Bcn | | PW Bcn | 2010 vs 2003-2005 | | 2011-2012 vs 2010 | |
| Variable | Ratio (95% CI) | p | (%) | Ratio (95% CI)* | p | Ratio (95% CI)* | p |
| Proteins | | | | | | | |
| DBL3X | 8.24 (4.85; 14) | <0.001 | 2 | 0.36 (0.25; 0.53) | <0.001 | 0.95 (0.69; 1.31) | 0.756 |
| DBL5ε | 8.5 (4.9; 14.75) | <0.001 | 0 | 0.32 (0.22; 0.48) | <0.001 | 0.96 (0.69; 1.33) | 0.796 |
| DBL6ε | 1.11 (0.82; 1.49) | 0.503 | 12 | 0.57 (0.46; 0.71) | <0.001 | 1.05 (0.88; 1.26) | 0.567 |
| AMA1 | 24.51 (18.53; 32.43) | <0.001 | 2 | 0.7 (0.59; 0.83) | <0.001 | 0.88 (0.73; 1.07) | 0.197 |
| MSP119 | 85.07 (46.72; 154.9) | <0.001 | 4 | 0.43 (0.28; 0.65) | <0.001 | 0.88 (0.60; 1.30) | 0.533 |
| Peptides | | | | | | | |
| p1 | 3.08 (2.22; 4.25) | <0.001 | 4 | 0.56 (0.45; 0.71) | <0.001 | 1.31 (1.07; 1.61) | 0.008 |
| p4 | 2.65 (1.79; 3.9) | <0.001 | 4 | 0.6 (0.46; 0.77) | <0.001 | 1.03 (0.81; 1.31) | 0.805 |
| p5 | 3.15 (2.2; 4.52) | <0.001 | 4 | 0.47 (0.36; 0.60) | <0.001 | 1.61 (1.30; 2.00) | <0.001 |
| p6 | 4.32 (2.96; 6.31) | <0.001 | 0 | 0.57 (0.44; 0.74) | <0.001 | 1.23 (0.97; 1.56) | 0.093 |
| p8 | 2.85 (1.92; 4.23) | <0.001 | 0 | 0.44 (0.34; 0.58) | <0.001 | 1.24 (0.98; 1.58) | 0.071 |
| p10 | 2.24 (1.65; 3.04) | <0.001 | 2 | 0.76 (0.61; 0.95) | 0.017 | 1.1 (0.91; 1.33) | 0.323 |
| p11 | 2.37 (1.78; 3.15) | <0.001 | 4 | 0.79 (0.65; 0.96) | 0.016 | 0.9 (0.75; 1.08) | 0.248 |
| p12 | 2.92 (2.18; 3.91) | <0.001 | 0 | 0.55 (0.45; 0.69) | <0.001 | 1.27 (1.07; 1.51) | 0.007 |
| p18 | 2.53 (1.74; 3.68) | <0.001 | 6 | 0.58 (0.45; 0.76) | <0.001 | 1.14 (0.91; 1.43) | 0.254 |
| p20 | 2.24 (1.64; 3.06) | <0.001 | 4 | 0.6 (0.49; 0.75) | <0.001 | 1.26 (1.04; 1.53) | 0.02 |
| p21 | 2.23 (1.68; 2.95) | <0.001 | 0 | 0.67 (0.56; 0.80) | <0.001 | 0.99 (0.83; 1.18) | 0.878 |
| p22 | 1.74 (1.31; 2.31) | <0.001 | 6 | 0.78 (0.65; 0.95) | 0.013 | 0.99 (0.83; 1.19) | 0.919 |
| p23 | 1.71 (1.33; 2.2) | <0.001 | 4 | 0.79 (0.66; 0.94) | 0.008 | 0.98 (0.84; 1.14) | 0.783 |
| p24 | 2.13 (1.63; 2.78) | <0.001 | 2 | 0.65 (0.54; 0.79) | <0.001 | 1.29 (1.09; 1.51) | 0.002 |
| p27 | 3.58 (2.71; 4.75) | <0.001 | 2 | 0.61 (0.51; 0.74) | <0.001 | 0.98 (0.82; 1.17) | 0.858 |
| p29 | 1.55 (1.1; 2.18) | 0.0118 | 14 | 0.74 (0.58; 0.94) | 0.015 | 1.24 (1.00; 1.52) | 0.046 |
| p33 | 2.5 (1.91; 3.28) | <0.001 | 0 | 0.76 (0.62; 0.92) | 0.005 | 1.18 (1.00; 1.40) | 0.053 |
| p35 | 2.29 (1.6; 3.27) | <0.001 | 10 | 0.65 (0.51; 0.83) | <0.001 | 1.28 (1.03; 1.59) | 0.025 |
| p36 | 3.03 (2.33; 3.95) | <0.001 | 2 | 0.56 (0.47; 0.67) | <0.001 | 1.14 (0.97; 1.33) | 0.107 |
| p37 | 2.53 (1.84; 3.49) | <0.001 | 0 | 0.66 (0.53; 0.81) | <0.001 | 1.24 (1.01; 1.51) | 0.04 |
| p38 | 2.7 (2.11; 3.46) | <0.001 | 4 | 0.66 (0.57; 0.77) | <0.001 | 0.98 (0.83; 1.14) | 0.776 |
| p39 | 3.01 (2.2; 4.1) | <0.001 | 0 | 0.53 (0.42; 0.66) | <0.001 | 1.15 (0.95; 1.39) | 0.149 |
| p42 | 2.67 (1.93; 3.7) | <0.001 | 14 | 0.67 (0.54; 0.84) | <0.001 | 1.16 (0.95; 1.43) | 0.147 |
| p44 | 2.32 (1.71; 3.15) | <0.001 | 0 | 0.67 (0.56; 0.82) | <0.001 | 1.05 (0.86; 1.27) | 0.644 |
| p46 | 2.07 (1.49; 2.89) | <0.001 | 2 | 0.67 (0.54; 0.84) | <0.001 | 1.17 (0.95; 1.44) | 0.137 |
| pcsp | 8.2 (4.84; 13.87) | <0.001 | 10 | 0.49 (0.34; 0.71) | <0.001 | 1.1 (0.80; 1.52) | 0.553 |

PW, pregnat women;
Mz, mozambique;
Bcn, Barcelona;
SeroPrev, seroprevalence;
CI, confidence interval
*Liner regression, adjusted by parity, age, treatment and HIV

Abbreviations: PW, pregnant women; Mz, mozambique; Bcn, Barcelona; SeroPrev, seroprevalence; CI, confidence interval

*Linear regression, adjusted by parity, age, treatment and HIV

Antibody levels were higher in pregnant Mozambican women than in pregnant Spanish women (N=49) never exposed to malaria (p<0.05 in all cases) with the only exception of DBL6ϵ (ratio [95% CI]=1.11 [0.82; 1.49]; p=0.503) (FIG. 1B, upper panel; Table 2A). Seroprevalences obtained among pregnant Spanish women were below 5% for the majority of antigens except for p18, p22, p29, p35, p42, pCSP and DBL6ϵ that were above (6% to 14%) (FIG. 1B, lower panel; Table 2A).

Antibody levels measured at delivery against all malaria antigens were able to mirror the sharp reduction in P. falciparum infection that occurred between 2003 and 2010. Among VAR2CSA peptides, level of antibodies against p8 show the highest decrease (adjusted ratio and 95% confidence interval [AR-CI_{95%}]=0.44 [0.34, 0.58]) immediately followed by antibodies against p5 (AR-CI_{95%}=0.47 [0.36, 0.60]) and antibodies against p11 show the lowest decrease (AR-CI_{95%}=0.79 [0.65, 0.96]); p<0.05 in all cases. The same ability to mimic the decrease was observed for IgG levels against DBL3x (AR-CI_{95%}=0.36 [0.25, 0.53]; p<0.001), DBL5ϵ (AR-CI_{95%}=0.32 [0.22, 0.48]; p<0.001), DBL6ϵ (AR-CI_{95%}=0.57 [0.46, 0.71]; p<0.001) and non-VAR2CSA P. falciparum antigens (AMA1: AR-CI_{95%}=0.70 [0.59, 0.83] and pCSP: AR-CI_{95%}=0.49 [0.34, 0.71]; p<0.001) (FIG. 2, lower panel; Table 2A).

IgG levels against 8 out of the 25 VAR2CSA peptides analyzed were able to mirror the slight increase in malaria prevalence from 2010 to 2011-2012 (AR-CI_{95%} ranged from 1.24 [1, 1.52] for p29 to 1.61 [1.30, 2] for p5; p<0.05 in all cases) and similar increases were observed against additional 2 peptides (p6: AR-CI_{95%}=1.23 [0.97, 1.56]; p=0.093; p8: AR-CI_{95%}=1.24 [0.98, 1.58; p=0.071) although not statistically significant. No significant increase in IgG levels against VAR2CSA recombinant domains and non-VAR2CSA antigens were observed during these 3 years (p>0.1 in all cases) (FIG. 2, upper panel; Table 2A).

Taking all together, 7 VAR2CSA peptides (p1, p5, p6, p8, p12, p20 and p37) were selected because were immunoreactive (mean antibody levels above BSA recognition), were recognized at higher levels by pregnant Mozambican women compared with pregnant Spanish women (seroprevalence below 5% among pregnant Spanish women) and antibody levels were able to mirror the decrease (from 2003 to 2010) and the slight increase (from 2010 to 2012) in malaria prevalence in Mozambique.

3. Selection of VAR2CSA Peptides Eliciting IgG Responses Rapidly Generated with a Limited Life-Time

IgGs against 25 VAR2CSA peptides, recombinant domains (DBL3X, DBL5ϵ, DBL6ϵ) and non-VAR2CSA antigens (AMA1, MSP1₁₉ and pCSP) were measured during pregnancy and at delivery in a total of 696 plasmas from 239 pregnant Mozambican women followed during pregnancy between 2011 and 2012.

Antibody levels measured at delivery against all malaria antigens were increased in women having at least one detected infection during pregnancy compared with women that infection was not detected during pregnancy (p<0.05 in all cases) (Table 2B).

TABLE 2B
Selection of VAR2CSA peptides eliciting IgG responses rapidly generated with limited life-time

| | Infection during pregnacy | | Parasite density | | Antibody dynamics | | |
|---|---|---|---|---|---|---|---|
| | Infection vs no-infection | | Low vs High | | Time to double | | Half-life |
| Variable | ratio (95% CI)* | p | ratio (95% CI)* | p | weeks (95% CI)** | p | weeks (95% CI)** |
| Proteins | | | | | | | |
| DBL3X | 8.83 (5.33; 14.62) | <0.001 | 0.81 (0.35; 1.88) | 0.621 | 20.77 (13.44; 45.70) | <0.001 | 51.97 (29.01; 249.53) |
| DBL5ε | 15.06 (8.29; 27.37) | <0.001 | 0.80 (0.28; 2.25) | 0.674 | 16.25 (10.91; 31.83) | <0.001 | 34.57 (21.64; 85.94) |
| DBL6ε | 3.13 (2.33; 4.21) | <0.001 | 1.12 (0.61; 2.05) | 0.709 | 27.77 (19.76; 46.72) | <0.001 | 42.91 (24.07; 197.33) |
| AMA1 | 1.8 (1.30; 2.50) | <0.001 | 1.18 (0.96; 1.46) | 0.119 | 91.65 (39.61; ∞) | 0.136 | 217.21 (96.77; ∞) |
| MSP1_19 | 3.69 (1.83; 7.46) | <0.001 | 2.55 (0.98; 6.63) | 0.061 | 31.69 (15.65; ∞) | 0.056 | 66.99 (36.04; 474.23) |
| Peptides | | | | | | | |
| p1 | 1.73 (122; 2.46) | <0.001 | 0.94 (0.48; 1.83) | 0.851 | 32.14 (20.42; 75.39) | <0.001 | 190.49 (50.84; ∞) |
| p4 | 1.92 (1.30; 2.84) | <0.001 | 0.67 (0.32; 1.42) | 0.305 | 59.31 (28.80; ∞) | 0.064 | 64.98 (32.08; ∞) |
| p5 | 2.15 (1.39; 3.31) | <0.001 | 0.69 (0.29; 1.66) | 0.414 | 23.24 (16.16; 41.38) | <0.001 | 69.3 (33.68; ∞) |
| p6 | 1.62 (1.11; 2.37) | 0.013 | 1.07 (0.53; 2.17) | 0.853 | 37.58 (21.77; 137.40) | 0.007 | 49.2 (25.89; 494.21) |
| p8 | 2.17 (1.46; 323) | <0.001 | 0.69 (0.28; 1.71) | 0.43 | 27.52 (17.91; 59.40) | <0.001 | 28.65 (19.61; 53.15) |
| p10 | 1.88 (1.30; 2.73) | <0.001 | 0.84 (0.42; 1.70) | 0.635 | 23.24 (16.38; 40.00) | <0.001 | 46.57 (25.20; 307.08) |
| p11 | 2 (1.37; 2.93) | <0.001 | 0.82 (0.36; 1.86) | 0.643 | 38.07 (22.08; 138.16) | 0.007 | 135.4 (41.50; ∞) |
| p12 | 1.98 (1.41; 2.80) | <0.001 | 0.77 (0.37; 1.61) | 0.495 | 34.37 (21.96; 79.10) | <0.001 | 46.08 (26.99; 157.30) |
| p18 | 2.24 (1.42; 3.53) | <0.001 | 0.99 (0.43; 2.27) | 0.981 | 26.03 (16.46; 62.27) | <0.001 | 32.86 (21.00; 75.54) |
| p20 | 1.73 (127; 2.36) | <0.001 | 0.95 (0.52; 1.73) | 0.874 | 24.16 (17.09; 41.21) | <0.001 | 84.28 (36.21; ∞) |
| p21 | 1.49 (1.06; 2.10) | 0.021 | 0.78 (0.41; 1.48) | 0.451 | 32.32 (20.64; 74.47) | <0.001 | 72.55 (29.80; ∞) |
| p22 | 1.52 (1.09; 2.11) | 0.014 | 0.93 (0.50; 1.76) | 0.835 | 31.52 (20.97; 63.35) | <0.001 | 120.71 (37.37; ∞) |
| p23 | 1.33 (1.01; 1.75) | 0.041 | 0.83 (0.50; 1.40) | 0.498 | 33.99 (21.84; 76.64) | <0.001 | 43.06 (22.94; 350.98) |
| p24 | 1.73 (1.27; 2.35) | <0.001 | 0.80 (0.39; 1.64) | 0.538 | 38.48 (22.50; 132.99) | 0.006 | 50.39 (25.28; 7873.64) |
| p27 | 1.38 (1.06; 1.79) | 0.016 | 1.01 (0.60; 1.70) | 0.972 | 55.77 (31.07; 271.91) | 0.014 | 116.82 (44.44; ∞) |
| p29 | 2.6 (1.62; 4.15) | <0.001 | 0.54 (0.22; 1.29) | 0.169 | 21.44 (14.57; 40.63) | <0.001 | 63.92 (30.39; ∞) |
| p33 | 1.66 (1.19; 2.31) | <0.001 | 0.77 (0.39; 1.48) | 0.431 | 34.42 (21.98; 79.39) | <0.001 | 57.08 (28.77; 3592.58) |
| p35 | 2.21 (1.38; 3.54) | <0.001 | 0.72 (0.33; 1.59) | 0.422 | 21.3 (14.48; 40.31) | <0.001 | 35.95 (19.65; 210.67) |
| p36 | 1.57 (1.18; 2.09) | <0.001 | 0.68 (0.38; 1.22) | 0.206 | 39.68 (23.46; 128.50) | 0.005 | 39.91 (23.76; 124.55) |
| p37 | 2.13 (1.39; 3.29) | <0.001 | 0.63 (0.28; 1.44) | 0.278 | 26.87 (17.92; 53.68) | <0.001 | 64.77 (30.62; ∞) |
| p38 | 1.73 (1.34; 2.24) | <0.001 | 0.87 (0.49; 1.54) | 0.642 | 29.75 (20.82; 52.07) | <0.001 | 62.42 (31.49; 3574.05) |
| p39 | 2.86 (1.96; 4.17) | <0.001 | 0.85 (0.41; 1.79) | 0.676 | 23.67 (15.86; 46.62) | <0.001 | 171.55 (45.06; ∞) |
| p42 | 1.94 (1.32; 2.84) | <0.001 | 0.89 (0.49; 1.59) | 0.693 | 43.34 (25.60; 141.17) | 0.005 | N/A |
| p44 | 1.61 (1.12; 2.32) | 0.011 | 0.67 (0.34; 1.31) | 0.246 | 31.95 (18.95; 101.86) | 0.004 | 50.13 (25.67; 1070.65) |
| p46 | 1.45 (1.03; 2.04) | 0.034 | 0.86 (0.41; 1.78) | 0.685 | 129.97 (38.68; ∞) | 0.406 | 35.55 (22.38; 86.37) |
| pcsp | 3.1 (1.75; 5.49) | <0.001 | 1.81 (0.75; 4.37) | 0.195 | 51.33 (22.30; ∞) | 0.132 | N/A |

| | Antibody dynamics | | Parity | | |
|---|---|---|---|---|---|
| | Sero-revertion | | MG vs PG | | SeroPrev |
| Variable | weeks (95% CI)** | p | ratio (95% CI)*** | p | Delivery % |
| Proteins | | | | | |
| DBL3X | 138.85 (77.49; 666.65) | 0.013 | 2.22 (1.10; 4.51) | 0.028 | 45 |
| DBL5ε | 122.28 (76.54; 304.00) | 0.001 | 3.72 (1.61; 8.59) | 0.002 | 50 |
| DBL6ε | 49.09 (27.54; 225.73) | 0.012 | 1.84 (1.22; 2.79) | 0.004 | 13 |
| AMA1 | 544.49 (242.56; ∞) | 0.115 | 1.2 (0.76; 1.9) | 0.439 | 89 |
| MSP1_19 | 385.42 (207.36; 2728.50) | 0.023 | 1.6 (0.6; 4.29) | 0.351 | 89 |
| Peptides | | | | | |
| p1 | 231.82 (61.86; ∞) | 0.476 | 1.15 (0.70; 1.88) | 0.574 | 23 |
| p4 | 84.24 (41.59; ∞) | 0.056 | 1.4 (0.81; 2.42) | 0.229 | 22 |
| p5 | 141.17 (68.62; ∞) | 0.064 | 1.46 (0.80; 2.68) | 0.219 | 26 |
| p6 | 77.44 (40.75; 777.82) | 0.03 | 1.18 (0.69; 2.01) | 0.543 | 19 |
| p8 | 65.61 (44.91; 121.70) | <0.001 | 1.35 (0.77; 2.35) | 0.293 | 26 |
| p10 | 78.63 (42.54; 518.42) | 0.021 | 1.54 (0.91; 2.58) | 0.107 | 17 |
| p11 | 209.31 (64.15; ∞) | 0.386 | 1.12 (0.66; 1.91) | 0.675 | 21 |
| p12 | 78.95 (46.25; 269.54) | 0.006 | 1.35 (0.84; 2.19) | 0.22 | 16 |
| p18 | 59.85 (38.24; 137.58) | <0.001 | 1.29 (0.68; 2.43) | 0.44 | 26 |
| p20 | 101.41 (43.57; ∞) | 0.14 | 1.51 (0.98; 2.32) | 0.065 | 21 |
| p21 | 133.6 (54.88; ∞) | 0.172 | 1.29 (0.80; 2.08) | 0.289 | 15 |
| p22 | 244.05 (75.55; ∞) | 0.38 | 1.31 (0.82; 2.07) | 0.261 | 17 |
| p23 | 57 (30.36; 464.65) | 0.026 | 1.15 (0.79; 1.69) | 0.461 | 15 |
| p24 | 90.94 (45.62; 14209.30) | 0.049 | 1.46 (0.95; 2.23) | 0.088 | 18 |
| p27 | 147.1 (55.96; ∞) | 0.229 | 1.22 (0.85; 1.76) | 0.279 | 18 |
| p29 | 129.91 (61.77; ∞) | 0.076 | 1.18 (0.61; 2.28) | 0.621 | 28 |
| p33 | 102.25 (51.54; 6435.16) | 0.046 | 1.21 (0.76; 1.93) | 0.416 | 18 |
| p35 | 54.92 (30.02; 321.81) | 0.018 | 1.36 (0.70; 2.63) | 0.364 | 24 |
| p36 | 44.41 (26.44; 138.60) | 0.004 | 1.44 (0.96; 2.15) | 0.078 | 17 |
| p37 | 145.45 (68.76; ∞) | 0.079 | 1.41 (0.77; 2.58) | 0.27 | 18 |
| p38 | 74.37 (37.51; 4258.27) | 0.046 | 1.1 (0.77; 1.58) | 0.595 | 17 |
| p39 | 281 (73.80; ∞) | 0.485 | 1.33 (0.78; 2.26) | 0.297 | 31 |
| p42 | N/A | 0.325 | 0.91 (0.53; 1.56) | 0.739 | 26 |
| p44 | 75.56 (38.69; 1613.79) | 0.04 | 1.15 (0.69; 1.93) | 0.575 | 21 |
| p46 | 78.13 (49.19; 189.80) | <0.001 | 1.18 (0.73; 1.90) | 0.496 | 19 |
| pcsp | N/A | 0.577 | 222 (1.00; 4.93) | 0.052 | 64 |

CI, confidence interval;
MG, multigravid;
PG, primigravid
*Linear regression adjusted by age, parity, treatment and HIV infection
**Log linear regression models adjusted by parity, age, treatment and HIV infection
***Linear regression adjusted by malaria infection, age, treatment and HIV infection

Abbreviations: CI, confidence interval; MG, multigravid; PG, primigravid

*Linear regression adjusted by age, parity, treatment and HIV infection

**Log linear regression models adjusted by parity, age, treatment and HIV infection

***Linear regression adjusted by malaria infection, age, treatment and HIV infection

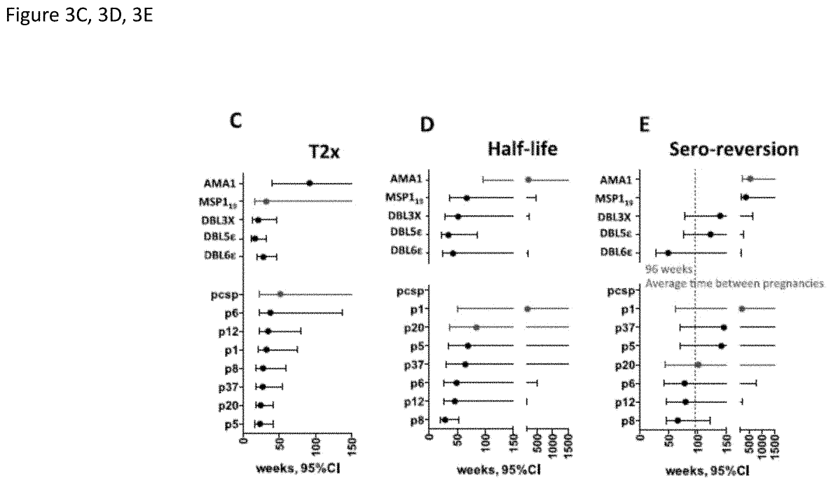

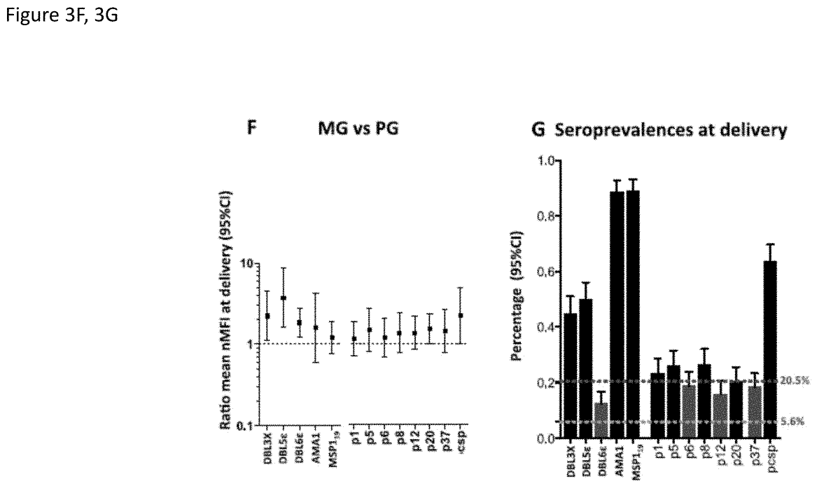

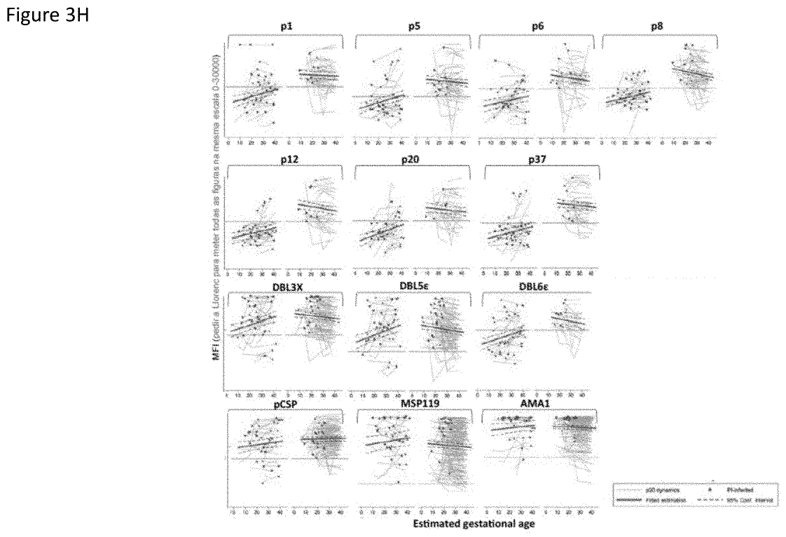

Among the 7 VAR2CSA peptides, IgG levels against p5 (AR-CI_{95%}=2.15 [1.39, 3.31]; p<0.001), p8 (AR-CI_{95%}=2.17 [1.46, 3.23]; p<0.001) and p37 (AR-CI_{95%}=2.13 [1.39, 3.29]; p<0.001) show the highest increase (FIG. 3A). Much high increase was observed for DBL3x (AR-CI_{95%}=8.83 [5.33, 14.62]; p<0.001) and DBL5ϵ (AR-CI_{95%}=15.06 [8.29, 27.37]; p<0.001) (FIG. 3A).

Different parasite densities (low: <200 genomes per microliter [n=36] and high: >200 genomes per microliter [N=31]) did not had an effect on antibody levels against all the antigens (P>0.05) independent of time of collection (FIG. 3B; Table 2B).

Estimates of time (in weeks) to double (T2x) the antibody levels (measured in 26 women experiencing infection on follow-up) obtained among the 7 peptides ranged from 23.24 (95% CI=16.16, 41.38; p=<0.001) for p5 to 37.58 (95% CI=21.77, 137.40; p=0.007) for p6. Among the recombinant domains, T2x were 20.77 (95% CI=13.44, 45.70; p<0.001) for DBL3X, 16.25 (95% CI=10.91, 31.83; p<0.001) for DBL5ϵ and 27.77 (95% CI=19.76, 46.72; p<0.001) for DBL6ϵ (FIG. 3C). Contrary to what observed for VAR2CSA-based antigens, the majority of the women were seropositive against non-VAR2CSA antigens at recruitment and follow-up (FIG. 3H) resulting in not statistically significant Tx2 for IgG levels against AMA1, MSP1₁₉ and pCSP (p>0.05).

Half-lives (T1/2) and time to sero-negativization (TSN) of antibody levels (in weeks) against each antigen were estimated in a group of seropositive women at recruitment that did not experienced P. falciparum infection on follow-up (sample size ranged from N=22 for DBL6ϵ to N=182 for MSP1₁₉). Fast antibody decline among the 7 peptides were obtained for p8 (T_{1/2}: 28.65 weeks, 95% CI=19.61; 53.15 and TSN: 65.61 weeks, 95% CI=44.91, 121.70; p<0.001), followed by p12 (T_{1/2}: 46.08 [26.99, 157.30] and TSN: 78.95 [46.25, 269.54]; p=0.006), and p6 (T_{1/2}: 49.2 [25.89; 494.21] and TSN: 77.44 [40.75, 777.82]; p=0.03) and then by p37 (T_{1/2}: 64.77 [30.62; ∞] and TSN: 145.45 [68.76, 00]; p=0.079) and p5 (T_{1/2}: 69.3 [33.68; cc] and TSN: 141.17 [68.62, ∞]; p=0.064), although not highly statistically significant for the last 2 peptides (FIG. 3D,E; Table 2B). The difference between half-life and sero-negativisation was higher in DBL3X (T_{1/2}: 51.97 [29.01, 249.53] and TSN: 138.85 [77.49, 666.65]; p=0.013) and DBL5ϵ (T_{1/2}: 34.57 [21.64, 85.94] and TSN: 122.28 [76.54; 304.00]; p=0.001) compared with peptides (FIG. 3D, E; Table 2B). Among the non-VAR2CSA antigens, only MSP1₁₉ showed half-life and sero-negativisation statistically significant (T_{1/2}: 66.99 [36.04, 474.23] and TSN: 385.42 [207.36, 2728.50]; p=0.023) (FIG. 3D, E; Table 2B; FIG. 3H).

Antibody levels measured at delivery did not increase with increasing parity of the women for 6 VAR2CSA peptides (p>0.1) with the only exception of p20 (AR-CI_{95%}=1.51 [0.98, 2.32]; p<0.065) that slight increase (FIG. 3F; Table 2B). A higher increase of antibody levels in multigravid was reported for DBL3X (AR-CI_{95%}=2.22 [1.10, 4.51]; p<0.028), and DBL5ϵ (AR-CI_{95%}=3.72 [1.61, 8.59]; p<0.002) in accordance with the long time to sero-negativisation estimated for these antigens (FIG. 3E, F; Table 2B).

Finally, seroprevalences at delivery were similar and not below the 21% prevalence of infection detected during pregnancy for p1 (23%), p5 (26%), p8 (26%) and p20 (21%) (FIG. 3G; Table 2B). Recombinant domains show seroprevalences 2 times high (45% for DBL3X and 50% for DBL5ϵ; exception of 12% for DBL6ϵ) and non-VAR2CSA antigens 4 times high (89% for MSP1₁₉ and AMA1, and 64% for pCSP) than prevalence of infection detected during pregnancy (FIG. 3G; Table 2B).

Taking all together, p5 and p8 were selected because: First, antibody responses were highly increased at delivery in women that experienced a detected infection in agreement with the short time to double the antibody levels estimated in relation to other antigens. Second, antibodies did not increase with increasing parity of the women in accordance with a half-life and time to sero-negativisation below the average time reported in Mozambique for a second pregnancy to occur; Third, the seroprevalence at delivery was not below but similar with the prevalence of infection detected during that particular pregnancy.

4. Performance of Selected VAR2CSA Peptides to Identify Differences on Malaria Exposure by Time and Space

The prevalence of malaria infection assessed by qPCR in peripheral and placental blood at delivery decreased from 26% in 2003-2005 to 2% in 2010 (adjusted odds ratio and 95% CI (AOR-CI_{95%})=0.05 [0.01, 0.16]; p<0.001) and slightly increased to 6% in 2011-2012 (AOR-CI₉₅%=3.71 [1.08, 12.78]; p=0.0379).

Table 3A shows (Sero)Prevalence of Plasmodium falciparum among Mozambican pregnant women at delivery, according to year.

TABLE 3A

| | 2003/4 | 2005 | 2010 | 2011 | 2012 | Decrease | | iHIV | iParity | Increase | | iHIV | iParity |
|---|---|---|---|---|---|---|---|---|---|---|---|---|---|
| variable | (%) | (%) | (%) | (%) | (%) | (OR)* | p-value | (p) | (p) | (OR)# | p-value | (p) | (p) |
| PCR | 27 | 25 | 2 | 6 | 6 | 0.05 (0.01; 0.16) | <0.001 | na | 0.886 | 3.71 (1.08; 12.78) | 0.0379 | na | 0.864 |
| comp5&8 | 34 | 52 | 20 | 30 | 38 | 0.27 (0.16; 0.46) | <0.001 | 0.28 | 0.317 | 1.74 (1.12; 2.71) | 0.0135 | 0.18 | 0.63 |
| p5 | 21 | 37 | 10 | 19 | 24 | 0.22 (0.12; 0.41) | <0.001 | 0.188 | 0.112 | 2.35 (1.32; 4.19) | 0.004 | 0.82 | 0.575 |
| p8 | 28 | 40 | 16 | 20 | 33 | 0.38 (0.22; 0.67) | 0.001 | 0.406 | 0.68 | 1.52 (0.94; 2.47) | 0.091 | 0.16 | 0.568 |
| pcsp | 48 | 57 | 36 | 40 | 46 | 0.43 (0.27; 0.69) | 0.004 | 0.475 | 0.125 | 1.19 (0.80; 1.78) | 0.3829 | 0.688 | 0.65 |
| DBL3x | 69 | 70 | 49 | 49 | 63 | 0.34 (0.21; 0.56) | <0.001 | 0.862 | 0.093 | 1.05 (0.71; 1.56) | 0.7888 | 0.127 | 0.558 |
| DBL5e | 69 | 69 | 49 | 51 | 60 | 0.35 (0.21; 0.57) | <0.001 | 0.88 | 0.233 | 1.1 (0.75; 1.63) | 0.6156 | 0.083 | 0.339 |
| DBL6e | 36 | 38 | 16 | 22 | 21 | 0.3 (0.18; 0.51) | <0.001 | 0.581 | 0.765 | 1.35 (0.83; 2.20) | 0.2234 | 0.635 | 0.822 |
| AMA1 | 99 | 98 | 93 | 89 | 92 | 0.24 (0.06; 0.93) | 0.039 | na | 0.671 | 0.58 (0.29; 1.16) | 0.1251 | 0.101 | 0.581 |
| MSP119 | 90 | 99 | 94 | 84 | 90 | 0.81 (0.31; 2.14) | 0.6715 | 0.605 | 0.044 | 0.37 (0.19; 0.74) | 0.0049 | 0.809 | 0.224 |

na, not aplicable because few pcr+ hiv+
*2010 vs 2003/5
#2011/12 vs 2010
iParity, parity interaction
iHIV, HIV interaction

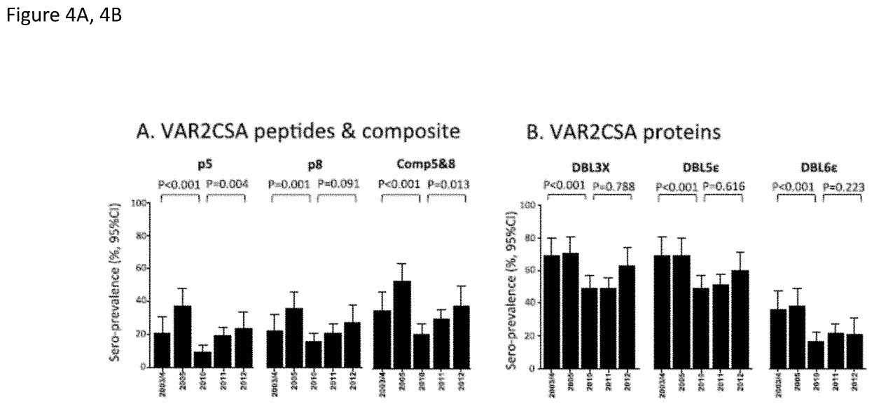

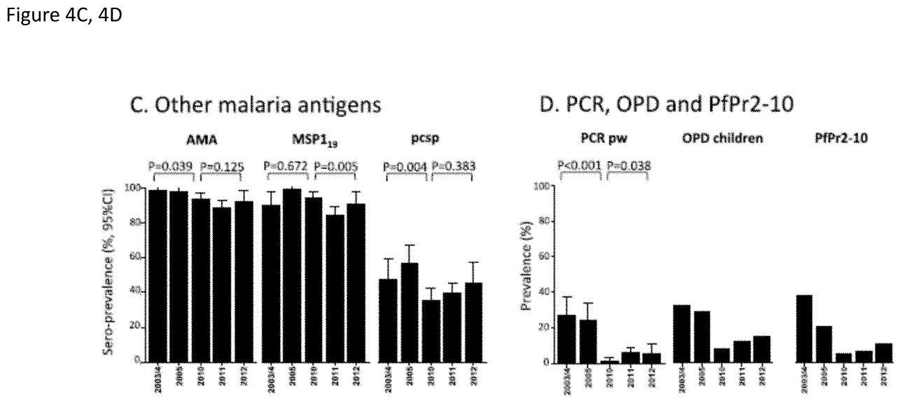

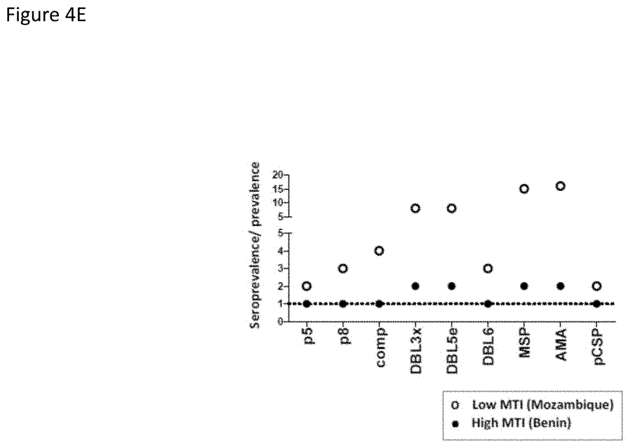

Seroprevalences against p5 and p8 significantly decreased from 29% and 34% in 2003-2005, to 10% and 16% in 2010 (p<0.001, adjusted) and slightly increased to 22% and 27% in 2012 (significant for p5 [p=0.004] and for p8 a similar increase was observed [p=0.091; adjusted] although not statistically significant), respectively (FIG. 4; Table 3A). On the other side, much higher seroprevalences were observed for recombinant domains (e.g. DBL5ϵ: 69% in 2003-2005, 49% in 2010 and 55% in 2011-2012) and non-VAR2CSA antigens (e.g. MSP1₁₉: 94% in 2003-2005, 94% in 2010 and 87% in 2011-2012). The capacity to mirror the decrease or increase by seroprevalences was not modified by HIV infection or parity of the women (p-interaction>0.05; with the only exception of parity effect on seroprevalences against MSP119) (Table 3A).

Prevalence of P. falciparum assessed by qPCR in peripheral and placental blood at delivery was 41% in Benin, 10% in Gabon and 6% in Mozambique among HIV-uninfected women, and 8% in Kenya and 3% in Mozambique among HIV-infected women. Similar trends were observed for seroprevalences against p5 and p8 among HIV-uninfected women: 41% and 42% in Benin; 23% and 25% in Gabon; 13% and 16% in Mozambique (p<0.001, adjusted), respectively. In HIV-infected women seroprevalences against p8 were high in Kenya than in Mozambique (17% vs 9%; p<0.001, adjusted) but no difference was observed for seroprevalences against p5 (6% in Kenya and Mozambique; p=0.894). On the other side, much higher seroprevalences in all countries were observed for recombinant domains (e.g. DBL5ϵ: 89% in Benin, 37% in Gabon and 45% in Mozambique among HIV-uninfected and 31% in Kenya and 36% in Mozambique among HIV-infected) and non-VAR2CSA antigens (e.g. MSP1₁₉: 99% in Benin, 84% in Gabon and 89% in Mozambique among HIV-uninfected and 88% in Kenya and 85% in Mozambique among HIV-infected).

Table 3B shows (sero)prevalences of Plasmodium falciparum among pregnant women, according to country.

TABLE 3B

| | HIV uninfected | | | | | | | HIV infected | | | | |
|---|---|---|---|---|---|---|---|---|---|---|---|---|
| | B | G | M | G | M | | | K | M | | | |
| variable | (%) | (%) | (%) | (OR)* | (OR)* | p-value | iParity (p)$ | (%) | (%) | M (OR)# | p-value | iParity (p) |
| PCR | 41 | 10 | 6 | 0.13 (0.06; 0.29) | 0.07 (0.04; 0.12) | <0.001 | 0.901 | 8 | 3 | 0.33 (0.15; 0.74) | 0.007 | 0.107 |
| comp5&8 | 61 | 33 | 24 | 0.31 (0.21; 0.47) | 0.21 (0.16; 0.28) | <0.001 | 0.904 | 21 | 14 | 0.58 (0.38; 0.88) | 0.011 | 0.128 |
| p5 | 41 | 23 | 13 | 0.4 (0.26; 0.62) | 0.2 (0.15; 0.28) | <0.001 | 0.715 | 6 | 6 | 0.96 (0.5; 1.82) | 0.894 | 0.311 |
| p8 | 42 | 25 | 16 | 0.5 (0.32; 0.76) | 0.29 (0.22; 0.39) | <0.001 | 0.378 | 17 | 9 | 0.45 (0.28; 0.73) | 0.001 | 0.181 |
| pcsp | 44 | 23 | 13 | 0.37 (0.24; 0.57) | 0.19 (0.14; 0.26) | <0.001 | 0.856 | 21 | 10 | 0.41 (0.26; 0.64) | <0.001 | 0.34 |
| DBL3x | 89 | 49 | 46 | 0.11 (0.07; 0.16) | 0.1 (0.08; 0.14) | <0.001 | 0.945 | 48 | 36 | 0.62 (0.45; 0.85) | 0.004 | 0.049 |
| DBL5e | 89 | 37 | 45 | 0.06 (0.04; 0.1) | 0.1 (0.07; 0.14) | <0.001 | 0.846 | 31 | 36 | 1.33 (0.96; 1.86) | 0.090 | 0.126 |
| DBL6e | 54 | 24 | 15 | 0.28 (0.18; 0.43) | 0.16 (0.12; 0.21) | <0.001 | 0.147 | 0 | 10 | na | na | na |
| AMA1 | 100 | 82 | 93 | na | na | na | na | 95 | 93 | 0.76 (0.40; 1.45) | 0.404 | na |
| MSP119 | 99 | 84 | 89 | 0.08 (0.04; 0.18) | 0.13 (0.07; 0.26) | <0.001 | 1 | 88 | 85 | 0.73 (0.46; 1.16) | 0.177 | 0.928 |

B, Benin;
G, Gabon;
M, Mozambique;
K, Kenya
*Benin as reference
#Kenya as reference
iParity, parity interaction
$parity interaction in Benin and Mozambique as reference of high and low transmission

The capacity of seroprevalences to distinguish high (Benin) from low (Mozambique) malaria transmission was not modified by parity with the only exception of seroprevalences against DBL3x in HIV-infected women (Table 3B). The difference between seroprevalences and qPCR prevalence at delivery was much higher in Mozambique (low transmission) than in Benin (high transmission) (FIG. 4E).

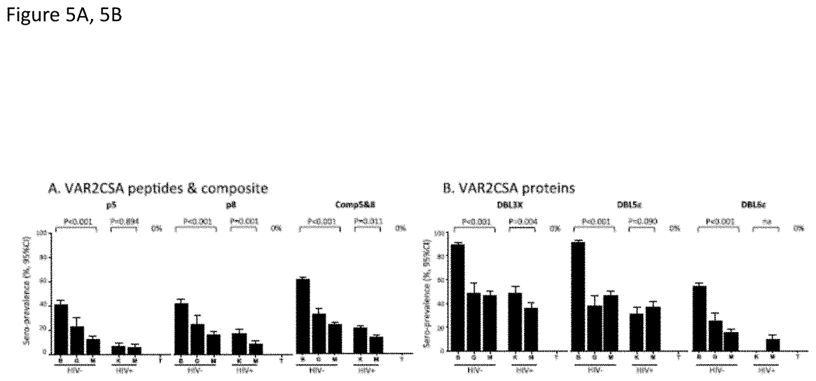

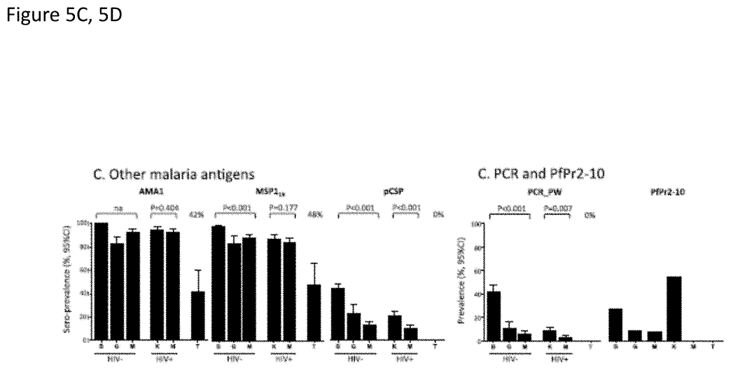

Moreover, pregnant women (all HIV-uninfected) living in an area from Tanzania where no malaria infection was observed were seronegative against VAR2CSA antigens and almost half of the women were seropositive against AMA1 (42%) and MSP119 (48%) (FIG. 5; Table 3B).

5. Performance of Selected VAR2CSA Peptides to Identify Recent Changes on Malaria Exposure

Seroprevalences against selected peptides were associated with reductions in exposure observed in HIV-uninfected women who received IPTp with MQ compared to those who received SP (p<0.05). Table 3C shows (sero)prevalences of Plasmodium falciparum among pregnant women, according to intermittent preventive treatment intervention group.

TABLE 3C

| | | HIV uninfected | | | | HIV infected | | | | |
|---|---|---|---|---|---|---|---|---|---|---|
| | MQ | SP | | | | PL | MQ | | | iParity |
| variable | (%) | (%) | SP (OR)* | p | iParity (p) | (%) | (%) | MQ (OR)# | p | (p) |
| PCR | 18 | 27 | 1.93 (1.28; 2.91) | 0.002 | 0.215 | 8 | 2 | 0.24 (0.10; 0.59) | 0.002 | 0.125 |
| comp5&8 | 44 | 51 | 1.35 (1.06; 1.70) | 0.013 | 0.964 | 19 | 15 | 0.76 (0.50; 1.15) | 0.198 | 0.079 |
| p5 | 28 | 34 | 1.33 (1.04; 1.7) | 0.022 | 0.793 | 7 | 5 | 0.66 (0.35; 1.27) | 0.215 | 0.684 |
| p8 | 30 | 36 | 1.33 (1.04; 1.69) | 0.021 | 0.765 | 14 | 10 | 0.68 (0.42; 1.1) | 0.115 | 0.078 |
| pcsp | 31 | 34 | 1.08 (0.84; 1.38) | 0.548 | 0.486 | 17 | 12 | 0.69 (0.44; 1.07) | 0.099 | 0.732 |
| DBL3x | 71 | 73 | 1.08 (0.81; 1.44) | 0.589 | 0.641 | 49 | 44 | 0.85 (0.61; 1.16) | 0.299 | 0.124 |
| DBL5e | 69 | 72 | 1.21 (0.90; 1.62) | 0.198 | 0.738 | 36 | 31 | 0.81 (0.58; 1.12) | 0.204 | 0.069 |
| DBL6e | 36 | 44 | 1.43 (1.12; 1.82) | 0.004 | 0.878 | 6 | 4 | na | na | 0.726 |
| AMA1 | 96 | 96 | na | na | na | 95 | 92 | 0.6 (0.32; 1.12) | 0.109 | na |
| MSP119 | 94 | 94 | 1.02 (0.63; 1.66) | 0.927 | 0.804 | 87 | 85 | 0.84 (0.54; 1.32) | 0.461 | 0.702 |

MQ, mefloquine;
SP, sulfadoxine-pyrimethamine;
PL, Placebo;
p, p-value
*MQ as reference
#Placebo as reference
iParity, parity interaction

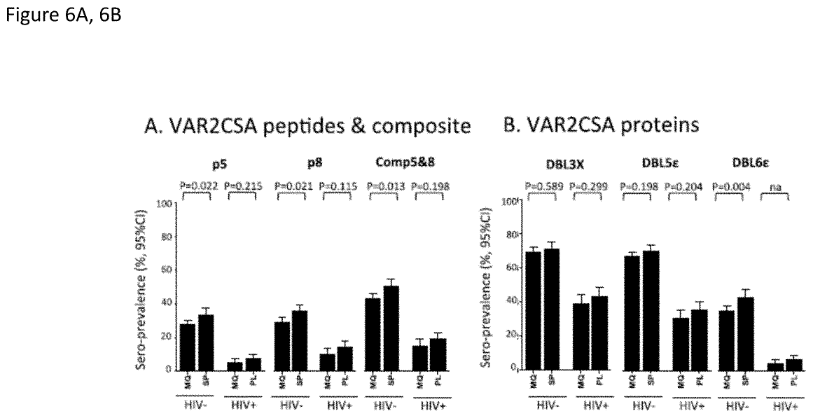

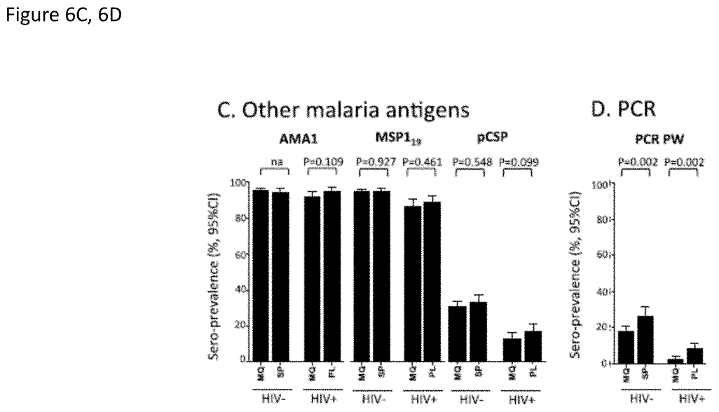

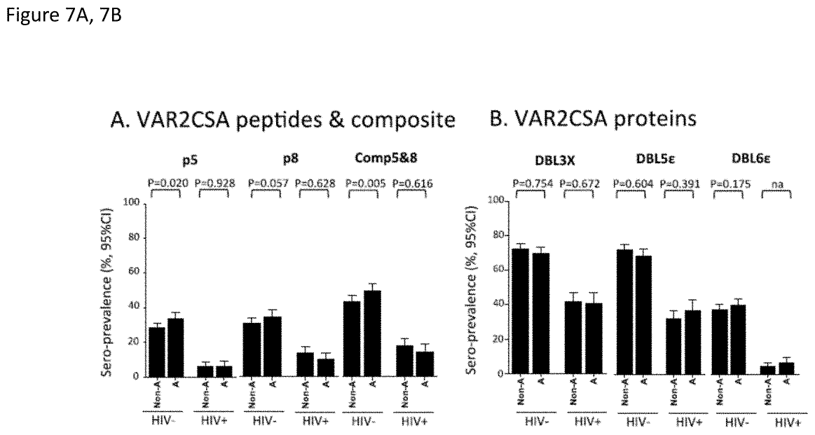

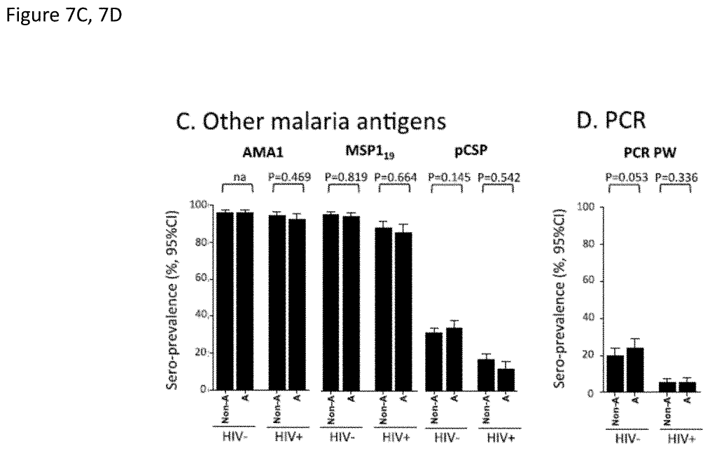

Similar differences were observed in HIV-infected women who received MQ compared to those who received placebo, although not statistically significant (FIG. 6; Table 3C). Anemic HIV-uninfected women show low seroprevalences against the selected peptides than non-anemic (p<0.05) (FIG. 7).

Table 3D shows (sero)prevalences of Plasmodium falciparum among pregnant women, according to anemia status.

TABLE 3D

| | | HIV uninfected | | | | | HIV infected | | | |
|---|---|---|---|---|---|---|---|---|---|---|
| | Non-A | A | | | iParity | Non-A | A | | | iParity |
| variable | (%) | (%) | A (OR)* | p | (p) | (%) | (%) | A (OR)* | p | (p) |
| PCR | 20 | 24 | 1.50 (0.99; 2.26) | 0.0529 | 0.047 | 5 | 5 | 1.49 (0.66; 3.36) | 0.336 | 0.993 |
| comp5&8 | 44 | 50 | 1.39 (1.11; 1.75) | 0.005 | 0.714 | 18 | 14 | 0.89 (0.56; 1.40) | 0.616 | 0.868 |
| p5 | 28 | 33 | 1.33 (1.05; 1.69) | 0.020 | 0.336 | 6 | 6 | 1.03 (0.52; 2.05) | 0.928 | 0.975 |
| p8 | 30 | 34 | 1.26 (0.99; 1.59) | 0.057 | 0.761 | 14 | 10 | 0.88 (0.52; 1.49) | 0.628 | 0.610 |
| pcsp | 31 | 34 | 1.19 (0.94; 1.51) | 0.145 | 0.440 | 17 | 12 | 0.86 (0.52; 1.41) | 0.542 | 0.384 |
| DBL3x | 72 | 70 | 0.96 (0.73; 1.26) | 0.754 | 0.013 | 42 | 40 | 1.08 (0.76; 1.52) | 0.672 | 0.808 |
| DBL5e | 71 | 68 | 0.93 (0.70; 1.23) | 0.604 | 0.012 | 32 | 36 | 1.17 (0.82; 1.65) | 0.391 | 0.900 |
| DBL6e | 37 | 40 | 1.18 (0.93; 1.49) | 0.175 | 0.677 | 5 | 7 | na | na | 0.467 |
| AMA1 | 96 | 96 | na | na | na | 94 | 92 | 0.79 (0.41; 1.51) | 0.469 | na |
| MSP119 | 95 | 94 | 0.95 (0.60; 1.51) | 0.819 | 0.721 | 87 | 85 | 0.90 (0.56; 1.45) | 0.664 | 0.700 |

Non-A, non anemia;
A, anemia;
p, p-value
*Non-A as reference
iParity, parity interaction

Parity did not modify the observed effect of IPTp or anemia in seroprevalences (Tables 3C and 3D). In general, differences among IPTp intervention groups and anemia status of pregnant women were not observed for seroprevalences against VAR2CSA recombinant domains neither against non-VAR2CSA antigens.

6. Surface Exposure, Epitope Prediction and Variability of Selected Peptides

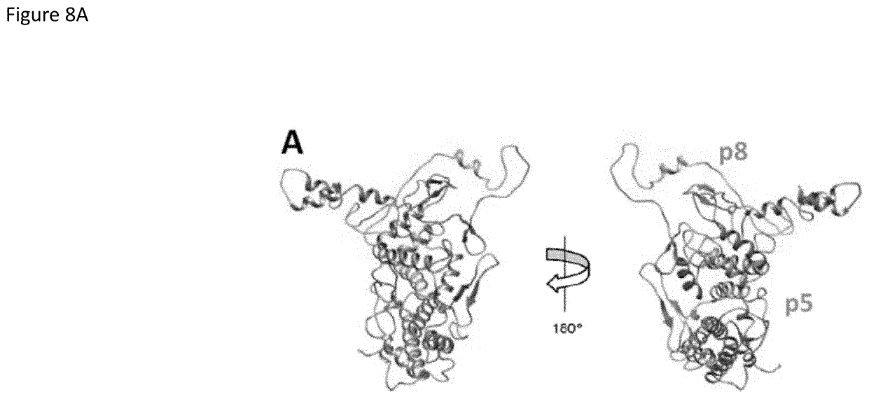

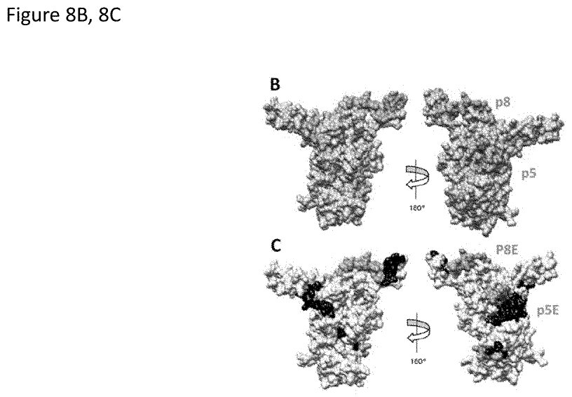

Mapping p5 and p8 amino acid segments on 3D structures of DBL1X-ID1 showed that p5 is localized in a less exposed area on the surface of the protein compared with p8 that is much more exposed to the surface (FIG. 8 A,B). Predicted B cell epitopes in p5 and p8 correspond to the more exposed areas of both peptides according to existing database records with specificity between 75% and 91% in BepiPred (FIG. 8C).

### CONCLUSION

VAR2CSA-serology based on antibodies against p5 and p8 detect a) malaria changes over time and space, b) recent changes of exposure resulting from IPTp interventions, c) absence of infection in a Tanzanian region where qPCR was negative. Moreover, seroprevalences based on p5 and p8 were similar with prevalence of infection detected by qPCR among pregnant women. This sero-surveillance tool could be used in pregnant women attending antenatal clinics to provide information about changes and monitor the absence of malaria transmission resulting from elimination activities.

1. A diagnostic test for monitoring infections by Plasmodium falciparum that is specific for pregnant women, comprising examining samples from the pregnant women, for the presence of antibodies to VAR2CSA.

2. The test of claim 1, wherein the samples comprise blood samples.

3. The test of claim 2, wherein said examining said samples comprises testing said samples for an antibody with a sufficiently short half-life to determine whether said infection occurred during a current pregnancy.

4. The test of claim 3, wherein the antibodies bind specifically to p5 and/or p8.


SEQUENCE LISTING (2 sequences)

SEQ ID NO: 1 - protein, 51 aa, P. falciparum
CRKCGHYEEKVPTKLDYVPQFLRWLTEWIEDLYREKQNLIDDMERHREECT

SEQ ID NO: 2 - protein, 48 aa, P. falciparum
DEVCNCNESEISSVGQAQTSGPSSNKTCITHSSIKANKKKVCKDVKLG